(19) United States
(12) Patent Application Publication
(10) Pub. No.: US 2009/0047722 A1
(43) Pub. Date: Feb. 19, 2009

(54) SYSTEMS, DEVICES, AND METHODS FOR BIOMASS PRODUCTION

(75) Inventors: Brian D. Wilkerson, Boise, ID (US); James C. Chen, Bellevue, WA (US); John Pulse, Woodinville, WA (US); Andrei Guschin, Issaquah, WA (US); Michael Weaver, Woodinville, WA (US)

Correspondence Address: SEED INTELLECTUAL PROPERTY LAW GROUP PLLC, 701 FIFTH AVE, SUITE 5400, SEATTLE, WA, 98104, US

(73) Assignee: BIONAVITAS, INC., Snoqualmie, WA (US)

(21) Appl. No.: 12/045,618

(22) Filed: Mar. 10, 2008

Related U.S. Application Data
(63) Continuation-in-part of application No. 11/608,527, filed on Dec. 8, 2006.
(60) Provisional application No. 60/749,243, filed on Dec. 9, 2005.
(60) Provisional application No. 60/773,183, filed on Feb. 14, 2006.

(51) Int. Cl. C12N 13/00; C12M 1/00; C12N 1/12

(52) U.S. Cl. 435/173.7; 435/292.1; 435/257.1

(57) ABSTRACT

Systems, devices, and methods for releasing one or more cell components from a photosynthetic organism. A bioreactor system is operable for growing photosynthetic organisms. Some of the methods include contacting the photosynthetic organism with an energy-activatable sensitizer, and activating the energy-activatable sensitizer, thereby releasing a cellular component from at least one of, for example, a membrane structure, tubule, vesicle, cisterna, organelle, cell compartment, plastid, or mitochondrion, associated with the photosynthetic organisms.

### CROSS-REFERENCES TO RELATED APPLICATIONS

This application is a continuation-in-part of U.S. patent application Ser. No. 11/608,527, filed Dec. 8, 2006, now pending which claims the benefit under 35 U.S.C. § 119(e) of U.S. Provisional Patent Application No. 60/749,243 filed Dec. 9, 2005, and U.S. Provisional Patent Application No. 60/773,183 filed Feb. 14, 2006.

### BACKGROUND

1. Field

The present invention generally relates to the field of bioreactors and, more particularly, to photobioreactor systems, devices, and methods using light sources to cultivate biomasses, photosynthetic organisms, living cells, biological active substances, and the like.

2. Description of the Related Art

A variety of methods and technologies exist for cultivating and harvesting biomasses such as, for example, mammalian, animal, plant, and insect cells, as well as various species of bacteria, algae, plankton, and protozoa. These methods and technologies include open-air systems and closed systems.

Algal biomasses, for example, are typically cultured in open-air systems (e.g., ponds, raceway ponds, lakes, and the like) that are subject to contamination. These open-air systems are further limited by an inability to substantially control the various process parameters (e.g., temperature, incident light intensity, flow, pressure, nutrients, and the like) involved in cultivating algae.

Alternatively, biomasses are cultivated in closed systems called bioreactors. These closed systems allow for better control of the process parameters, but are often more costly to setup and operate. In addition, these closed systems are limited in their ability to provide sufficient light to sustain dense populations of photosynthetic organisms cultivated within.

Biomasses have many beneficial and commercial uses including, for example, uses as pollution control agents, fertilizers, food supplements, cosmetic additives, pigment additives, and energy sources just to name a few. For example, algal biomasses are used in wastewater treatment facilities to capture fertilizers. Algal biomasses are also used to make biofuels.

Biofuels, such as biodiesel, can be used in existing diesel and compression ignition applications, where little or no modification to the engines and/or fuel delivery system is necessary. Biofuels are typically non-toxic and biodegradable; hence they provide an environmentally safe and cost-effective alternative fuel. The use of biofuels can help reduce pollution, as well as the environmental impacts of drilling, pumping, and transporting fossil-based diesel fuels.

Biofuels are already in use by some companies and governmental agencies, such as the U.S. Post Office, the Army and Air Force, the Department of Forestry, the General Services Administration, and the Agricultural Research Services. Some transit systems and school bus systems throughout the U.S. have also begun to use biofuel. Construction companies, in particular, stand to benefit tremendously from biofuel usage because most construction equipment is diesel-powered, for example cement trucks, dump trucks, bulldozers, spreaders, front loaders, cranes, backhoes, graders, and all sizes of generators. In addition, biofuel can be used in other industries such as in agricultural, farming, power plants, mining, railroad, and/or marine applications.

Because of their generally non-toxic and biodegradable nature, biofuels can also be useful in marine environments for applications other than powering a diesel-powered marine engine. For example, biofuel can be used for oil spill clean-ups in the ocean and to clean the wildlife and plant life affected by these spills. Biofuels may also be useful as solvents to remove paint, or clean out sludge from tanks used to store petroleum-based product. Further, biofuels have useful lubricant properties and can be used in a variety of machines. When used in diesel-powered engines, for example, the lubricity features of biofuels can extend the operational life of diesel-powered engines.

Typical bioreactors used for growing, for example, photosynthetic organism employ a constant intensity light source. One factor for cultivating biomasses (e.g., algae) in photobioreactors is providing and controlling the light necessary for the photosynthetic process. If the light intensity is too high or the exposure time too long, growth of the algae is inhibited. Moreover, as the density of the algae cells in the bioreactor increases, algae cells closer to the light source reduce the amount of light that reaches those algae cells that are further away from the light source.

Commercial acceptance of bioreactors is dependent on a variety of factors such as, for example, cost to manufacture, cost to operate, reliability, durability, and scalability. Commercial acceptance of bioreactors is also dependent on their ability to increase biomass production, while decreasing biomass production cost. Therefore, it may be desirable to have novel approaches for supplying light to a bioreactor and for sustaining the photosynthetic processes of a biomass cultivated within a reactor.

The present disclosure is directed to overcome one or more of the shortcomings set forth above, and/or provide further related advantages.

### BRIEF SUMMARY

In one aspect, the present disclosure is directed to a bioreactor system for cultivating photosynthetic organisms. The bioreactor system includes a container and a first lighting system.

The container includes an exterior surface and an interior surface. In some embodiments, the interior surface defines an isolated space configured to retain a plurality of photosynthetic organisms and cultivation media.

The first lighting system includes one or more light-emitting substrates received in the isolated space of the container each having a first surface and a second surface opposite to the first surface. The one or more light-emitting substrates are configured to supply a first amount of light from the first surface and a second amount of light from the second surface to at least some of a plurality of photosynthetic organisms retained in the isolated space.

In another aspect, the present disclosure is directed to a method of operating a bioreactor system for providing light energy to a substantial portion of a plurality of photosynthetic organisms in liquid growth media within a bioreactor.

The method includes providing a plurality of photosynthetic organisms and a cultivation media to an isolated space defined by an interior surface of a bioreactor containment structure. The method may include vertically or horizontally mixing the photosynthetic organisms included in the liquid growth media.

The method may further include operating a plurality of light-energy-supplying substrates received within the isolated space of the bioreactor. In some embodiments, each of the light-energy-supplying substrates comprises a first side and a second side opposite to the first side. In some embodiments, the first and second sides include one or more light-energy-supplying elements that form part of a light-energy-supplying area.

In another aspect, the present disclosure is directed to a photosynthetic biomass cultivation system. The photosynthetic biomass cultivation system includes a bioreactor and a controller. The controller is configured to automatically control at least one process variable associated with cultivating a photosynthetic biomass.

The bioreactor includes a structure having an exterior and interior surface, and a lighting system. In some embodiments, the interior surface defines an isolated space configured to retain the photosynthetic biomass suspended in cultivation media. The lighting system may include one or more light-emitting elements including a light-emitting area received in the isolated space of the structure. In some embodiments, the light-emitting area forms part of a light-emitting-area to reactor-volume interface.

In another aspect, the present disclosure is directed to a bioreactor system configured to adjust a light exposure of photosynthetic organisms located within the bioreactor. The bioreactor includes at least a first and second level for supporting a first and second surface layer of photosynthetic organisms, respectively. In some embodiments, the first level is physically separated from the second level. The bioreactor also includes a lighting system arranged to direct a first amount of light towards the first surface layer of photosynthetic organisms and further arranged to direct a second amount of light towards the second surface layer of photosynthetic organisms.

In another aspect, the present disclosure is directed to a method for increasing a ratio of light-emitting area to a photobioreactor volume of a photobioreactor. The method includes directing an effluent stream to the photobioreactor, which comprises a structure having an inner surface defining a photobioreactor volume.

The method further includes separating the effluent stream to direct one portion of the effluent stream to a first region of the photobioreactor holding a first amount of algae, and to direct another portion of the effluent stream to a second region of the photobioreactor holding a second amount of algae.

The method may also include directing light from a light source toward at least some of the algae in the photobioreactor to encourage a photosynthetic reaction in the algae, the light source comprising one or more light-emitting elements including a light-emitting area, the light-emitting area forming part of a light-emitting-area to photobioreactor-volume interface.

In another aspect, the present disclosure is directed to a bioreactor systems, devices, and methods for producing biofuel from algae. The system includes a bioreactor vessel, a control system, and a light source.

The bioreactor system includes a lighting system arranged to direct an amount of light on at least some algae located within the bioreactor vessel, the algae and lighting system respectively oriented within the bioreactor vessel to increase a photosynthetic process of the algae.

The control system is configured to monitor and/or control at least one environmental condition within the bioreactor vessel. In some embodiments, the light source is optically coupled to the lighting system.

In another aspect, the present disclosure is directed to a method of cultivating algae in a bioreactor. The method includes placing a first species and a second species of algae together in a portion of the bioreactor, wherein the first species includes a first light absorption capacity and the second species includes a second light absorption capacity. The method further includes controllably directing light toward the first and second species of algae.

In yet another aspect, the present disclosure is directed to a bio-system for extracting lipid from algae. The system includes a bioreactor, a control system, a light source, an extraction system, an inlet, and an outlet.

The bioreactor includes a lighting system arranged to direct an amount of light on at least some algae located within the bioreactor, the algae and lighting system respectively oriented within the bioreactor to increase a photosynthetic process of the algae. The bioreactor further includes a control system coupled to the bioreactor to monitor and/or control at least one environmental condition within the bioreactor.

The light source is optically coupled to the lighting system. The extraction system is operable to extract, for example, lipid, a medical compound, and/or a labeled compound from the algae from at least some of the algae. The inlet is coupled to the bioreactor, and configured to receive an effluent stream. The outlet is operable to discharge at least some algae. In some embodiments, the outlet is coupled to the extraction system to direct at least some algae thereto.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1A is a top front isometric view of a bioreactor according to one illustrated embodiment.

FIG. 1B is a functional block diagram showing a bioreactor system according to one illustrative embodiment.

FIG. 2 is an exploded view of a bioreactor according to one illustrated embodiment.

FIG. 3 is an exploded view of a bioreactor according to one illustrated embodiment.

FIG. 4 is a top front exploded cross-sectional view of a bioreactor according to one illustrated embodiment.

FIG. 5 is top front isometric view of a light system assembly and a sparging system for a bioreactor according to one illustrated embodiment.

FIG. 6 is top front isometric view of a light-emitting substrate for a bioreactor according to one illustrated embodiment.

FIG. 7 is a schematic view of a bioreactor according to one illustrated embodiment.

FIG. 8 is a schematic view of a lighting system for a bioreactor according to one illustrated embodiment.

FIG. 9 is a flow diagram of a method for providing light energy to a substantial portion of a plurality of photosynthetic organisms in liquid growth media within a bioreactor according to one illustrated embodiment.

FIG. 10 is a flow diagram of a method for increasing a ratio of light-emitting-area to a photobioreactor-volume interface of a photobioreactor according to one illustrated embodiment.

FIG. 11 is a cross-sectional side view of an open bioreactor filled with biomass producing material according to one illustrated embodiment.

FIG. 12 is a plan view of a portable open bioreactor according to one illustrated embodiment.

FIG. 13 is a side elevational view of the bioreactor of FIG. 12.

FIG. 14 is a cross-sectional isometric view of the bioreactor of FIG. 12 taken along the line 14-14.

FIG. 15 is a top front isometric view of a bioreactor, in the form of an open air reservoir comprising a plurality of light-emitting substrates, according to one illustrated embodiment.

FIG. 16 is a top front isometric view of a bioreactor, in the form of an open air reservoir comprising a plurality of light-emitting substrates, according to one illustrated embodiment.

FIG. 17 is a cross-sectional side view of an open bioreactor according to one illustrated embodiment.

FIG. 18 is a cross-sectional side view of a modified open bioreactor including an environment controlling structure according to one illustrated embodiment.

FIG. 19 is a cross-sectional side view of an open bioreactor including an environment controlling structure according to one illustrated embodiment.

FIG. 20 is a top front isometric view of an environment controlling structure according to one illustrated embodiment.

FIG. 21 is a cross-sectional side view of an open bioreactor including the environment controlling structure of FIG. 20 according to one illustrated embodiment.

FIG. 22 is a top front isometric view of a bioreactor, in the form of a modified open air reservoir comprising a plurality environment controlling structures according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with bioreactors, the transmission of effluent streams into and out of a bioreactor, the photosynthesis and lipid extraction processes of various types of biomass (e.g., algae and the like), fiber optic networks to include optical switching devices, light filters, solar collector systems to include solar array cells and solar collector mechanisms, methods of monitoring and harvesting a biomass (e.g., algae, and the like) to extract oil for biofuel purposes and/or convert a treated biomass (e.g., algae, and the like) to feedstock may not have been shown or described in detail to avoid unnecessarily obscuring the description.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment,” or “in some embodiments” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a bioreactor including “a light source” includes a single light source, or two or more light sources. It should also be noted that the term “or” is generally employed in its sense including “and/or” unless the content clearly dictates otherwise.

The term “bioreactor” as used herein and the claims generally refers to any system, device, or structure capable of supporting a biologically active environment. Examples of bioreactors include fermentors, photobioreactors, stir-tank reactors, airlift reactors, pneumatically mixed reactors, fluidized bed reactors, fixed-film reactors, hollow-fiber reactors, rotary cell culture reactors, packed-bed reactors, macro and micro bioreactors, and the like, or combinations thereof.

In some embodiments, the term bioreactor refers to a device or system for growing cells or tissues in the context of cell culture, such as the disposable chamber or bag, called a CELLBAG®, made by Panacea Solutions, Inc. and usable with systems developed by Wave Biotechs, LLC. In a further embodiment, the bioreactor can be a specially designed landfill for rapidly growing, transforming, and/or degrading organic structures. In yet a further embodiment, the bioreactor comprises a sphere and a mirror located outside of the sphere, wherein the shape of the sphere maximizes a surface to volume ratio of the algae contained therein and a waveguide for providing light from a light source, such as sunlight, into the sphere. Further examples of bioreactors include open-air systems such as ponds, raceway ponds, lakes, natural reservoirs, and the like, as well as regular and irregular shaped structures capable of sustaining biomass growth.

Accordingly, a bioreactor may be a closed or open system, but in certain embodiments will have the light sources described herein. In some embodiments, the two or more bioreactors may be coupled together to form a multi-reactor system. In further embodiments, the two or more bioreactors may be coupled in parallel and/or in series.

The term “biomass” as used herein and the claims generally refers to any biological material. Examples of a “biomass” include photosynthetic organisms, living cells, biological active substances, plant matter, living and/or recently living biological materials, and the like. Further examples of a “biomass” include mammalian, animal, plant, and insect cells, as well as various species of bacteria, algae, plankton, and protozoa.

The headings provided herein are for convenience only and do not interpret the scope or meaning of the claimed invention.

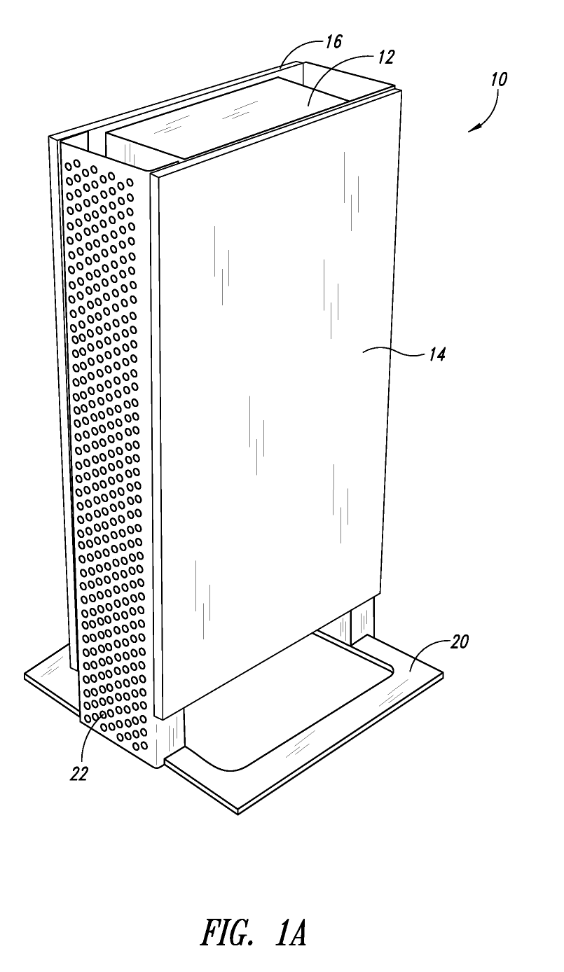

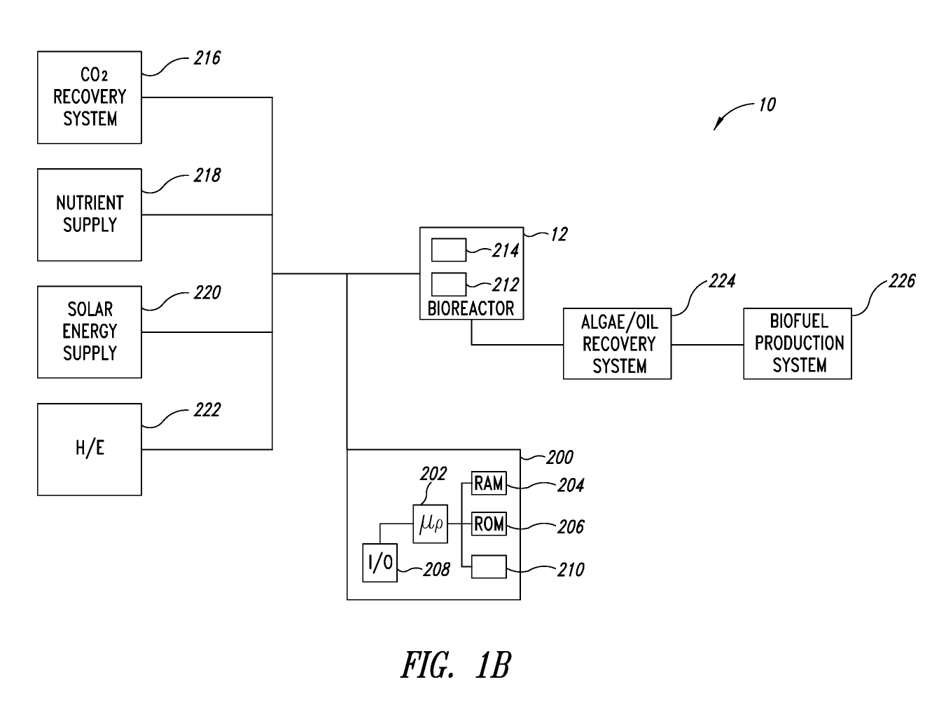

FIG. 1A shows an exemplary bioreactor system 10 for cultivating photosynthetic organisms. The bioreactor system 10 includes a bioreactor 12, housing structures 14, 16, and a support structure 20. The bioreactor system 10 may further include a side structure 22.

Referring to FIG. 1B, the bioreactor system 10 may further include a control system 200 operable to control the voltage, current, and/or power delivered to the bioreactor 12, as well as automatically control at least one process variable and/or a stress variable that alters or affects the growth and/or development of an organism (e.g., changing stress variable to induce nutrient deprivation, nitrogen-deficiency, silicon-deficiency, pH, CO₂ levels, oxygen levels, degree of sparging, or other conditions that affect growth and/or development of an organism). In some embodiments, the bioreactor 12 may operate under strict environmental conditions that require controlling one or more process variables associated with cultivating and/or growing a photosynthetic biomass. For example, the bioreactor system 10 may include one or more sub-systems for controlling gas flow rates (e.g., air, oxygen, CO₂, and the like), effluent streams, temperatures, pH balances, nutrient supplies, other organism stresses, and the like.

The control system 200 may include one or more controllers 202, for example, microprocessors, digital signal processor (DSPs) (not shown), an application-specific integrated circuits (ASICs) (not shown), field programmable gate arrays (FPGAs) (not shown), and the like. The control system 200 may also include one or more memories, for example, random access memory (RAM) 204, read-only memory (ROM) 206, and the like, coupled to the controllers 202 by one or more busses. The control system 200 may further include one or more input devices 208 (e.g., a keypad, touch-screen display, and the like). The control system 200 may also include discrete and/or an integrated circuit elements 210 to control the voltage, current, and/or power. In some embodiments, the control system 200 is configured to control at least one of light intensity, illumination intensity, a light-emitting pattern, a peak emission wavelength, an on-pulse duration, and a pulse frequency associated with one or more light-emitting substrates 34 based on a measured optical density.

The bioreactor system 10 may further include a variety of controller systems 200, sensors 212, as well as mechanical agitators 214, and/or filtration systems, and the like. These devices may be controlled and operated by the central control system 200. In some embodiments, the one or more sensors 212 may be operable and/or configured to determine at least one of a temperature, pressure, light intensity, optical density, opacity, gas content, pH, fluid level, sparging gas flow rate, salinity, fluorescence, absorption, mixing, and/or turbulence. The controller 200 may be configured to control at least one of an illumination intensity, illumination pattern, peak emission wavelength, ON-pulse duration, and/or pulse frequency based on a sensed temperature, pressure, light intensity, optical density, opacity, gas content, pH, fluid level, sparging gas flow rate, salinity, fluorescence, absorption, mixing, and/or turbulence.

The bioreactor system 10 may also include sub-systems and/or devices that cooperate to monitor and possibly control operational aspects such as the temperature, salinity, pH, CO₂ levels, O₂ levels, nutrient levels, and/or a light supply, and the like. In some embodiments, the bioreactor system 10 may include the ability to increase or decrease each aspect or parameter individually or in any combination, for example, temperature may be raised or lowered, gas (e.g., CO₂, O₂, etc.) levels may be raised or lowered, pH, nutrient levels, light, may be raised or lowered. The light can be natural or artificial. Some general lighting control aspects include controlling the duration that the light operates on portions of, for example, an algal mass in the bioreactor 12, cycling the light (to include periods of light and dark), for example artificial light, to extend the growth of the algae past daylight hours, controlling the wavelength of the light, controlling the lighting patterns, and/or controlling the intensity of the light. Lighting control may also include controlling one or more filters, operatives, masks, shades, and/or levers, particularly where the light is natural.

The bioreactor system 10 may further include a carbon dioxide recovery system 216 for recovering, treating, extracting, utilizing, scrubbing, cleaning, and/or purifying a carbon dioxide supply from, for example, flue gas of an industrial source (e.g., an industrial plant, an oil field, a coal mine, and the like).

The bioreactor system 10 may further include one or more nutrients supply systems 218, solar energy supply systems 220, and heat exchange systems 222.

The nutrients supply systems 218 may include, or be part of, one or more effluent and/or nutrient streams. An effluent is generally regarded as something that flows out or forth, like a stream flowing out of a body of water. For example, this includes, but is not limited to, discharged wastewater from a waste treatment facility, brine wastewater from desalting operations, and/or coolant water from a nuclear power plant. In the context of algae cultivation, an effluent stream may contain nutrients to feed algae present inside and/or outside of a bioreactor 12. In one embodiment, the effluent stream includes biological waste or waste sludge from a waste treatment facility (e.g., sewage, landfill, animal, slaughterhouse, toilet, outhouse, portable toilet waste, and the like). Such an effluent stream (including the CO₂ produced by the bacteria within such waste) can be directed to the algae, where the algae remove nitrogen, phosphate, and carbon dioxide (CO₂) from the stream. In another embodiment, the effluent stream comprises flue gases from power plants. The algae remove the CO₂ and various nitrogen compounds (NOx) from the flue gases. In each of the foregoing embodiments, the algae use the CO₂, in particular, for the process of photosynthesis. The oxygen produced by the algae during the photosynthetic process could be utilized to, for example, promote further bacterial growth and CO₂ production in a waste effluent stream. Furthermore, it is understood that the effluent streams can be seeded with a variety of additional nutrients and/or biological material to stimulate and enhance the growth rate, photosynthetic process, and overall cultivation of the algae.

The solar energy supply systems 220 may collect and/or supply sunlight, as well as direct light into the bioreactor 12. In some embodiments, solar energy supply systems 220 include a solar energy collector and a solar energy concentrator including a plurality of optical elements configured and positioned to collect and concentrate sun light.

The heat exchange system 222 typically controls and/or maintains a constant temperature within the bioreactor 12. For example, temperature may be lowered to stress the algae to promote oil production, etc., at end of a growth cycle. In some embodiments, the heat exchange system 222 and the control system 200 operate to maintain a constant temperature in the bioreactor 12 to sustain a bioprocess within.

The bioreactor system 10 may further include a biomass and/or oil recovery system 224, and a biofuel production system 226.

The biomass and/or oil recovery system 224 may take the form of an algae oil recovery system and may further include an extraction system, such as a press device or a centrifuge device to extract, for example, lipid, a medical compound, and/or a labeled compound from photoorganisms (e.g., algae, and the like). Various methods and techniques may be used for causing photoorganisms to produce medical compounds and/or labeled compounds (e.g., isotopically labeled compounds, and the like).

The extraction system may be located within or outside of the bioreactor 12. Additionally or alternatively, the extraction system may comprise an extractant selected from chemical solvents, supercritical gases or liquids, hexane, acetone, liquid petroleum products, and primary alcohols. In other embodiments, the extraction system includes a means for genetically, chemically, enzymatically or biologically extracting, or facilitating the extraction of, lipid from the algae.

In some embodiments, a conversion system may be operably coupled to the extraction system to receive the lipid and convert the lipid to biofuel. In one embodiment, the conversion system includes a transesterification catalyst and an alcohol. In other embodiments, the conversion system includes an alternate means for genetically, chemically, enzymatically, or biologically converting the lipid to biofuel.

In some embodiments, various enzymes may be utilized to break down the algal cell structure prior to extraction, thereby facilitating the subsequent extraction acts, e.g., minimizing the energy required in a physical extraction process such as a pressing or centrifuging.

The biofuel production system 226 may include various technologies for processing and/or refining biofuel from biomasses. For example, a catalytic cracking process can be used to produce other desirable fuel products and/or bi-products. Catalytic cracking breaks the complex hydrocarbons in the biofuel into simpler molecules to create a higher quality and greater quantity of a lighter, more desirable fuel product while also decreasing an amount of residuals in the biofuel. The catalytic cracking process rearranges the molecular structure of hydrocarbon compounds in the biofuel to convert heavy hydrocarbon feedstock into lighter fractions such as kerosene, gasoline, LPG, heating oil, and petrochemical feedstock.

For example, catalytic cracking is a process whereby catalytic material facilitates the conversion of the heavier hydrocarbon molecules into lighter products. The catalytic cracking process may be advantageous over thermal cracking processes because the yield of improved-quality fuels can be achieved under much less severe operating conditions than in thermal cracking, for example. The three types of catalytic cracking processes are fluid catalytic cracking (FCC), moving-bed catalytic cracking, and Thermofor catalytic cracking (TCC). The catalytic cracking process is very flexible, and operating parameters can be adjusted to meet changing product demand. In addition to cracking, catalytic activities include dehydrogenation, hydrogenation, and isomerization as described in, for example, U.S. Pat. No. 5,637,207.

Biodiesels and the production of biodiesels from, for example, algae can be used in a variety of applications. Such applications include the production of biodiesel and subsequent refinement to other fuels, including those that could be used as, or as a component of, jet fuels (e.g., kerosene). Such production could occur using catalytic cracking or any other known process for generating such fuels from the biofuels produced by algae. In one embodiment, such refining occurs as part of the same system used to extract the biofuels from the algae. In another embodiment, the biofuels are transported by truck, train, pipe, or other means to a second location where refining of the biofuel into other fuels such as those noted above occurs.

In some embodiments, the bioreactor system 10 takes the form of a bio-system configured to produce biofuel from algae. The bio-system includes a bioreactor 12 with a lighting system that is arranged to direct an amount of light on at least some algae located within the bioreactor 12. The algae can be brought into the bioreactor 12 via an effluent stream or the algae may be present within the bioreactor 12 prior to effluent introduction or may be seeded prior to effluent or nutrient stream introduction, concurrently therewith or subsequently. At least one or more filters can be positioned in the bioreactor 12 to filter non-algae type particulates from the effluent stream and/or separate the algae based on some characteristic or physical property of the algae.

The lighting system may be configured within the bioreactor 12 to increase the photosynthetic rate of the algae, and thus increase the yield of lipids from the algae. The bio-system may further include the control system 200 coupled to and/or located within the bioreactor 12 to monitor and/or control at least one environmental condition within the bioreactor 12, for example the temperature, humidity, effluent stream flow rate, and the like. In some embodiments, the control system 200 controls one or more sensors 212 (e.g., temperature sensor) located within a first region of the bioreactor 12. In some embodiments, an optical density or opacity measurement device measures the specific gravity and/or concentration of at least some of the algae just before it enters, or just after it enters, the bioreactor 12.

A light source is optically coupled to the lighting system. In one embodiment, the light source is a plurality of LEDs that direct artificial light toward at least some of the algae. In another embodiment, the light source is a solar collector that collects sunlight. The solar collector is coupled to the lighting system, which comprises a network of fiber optic waveguides and optical switches to route, guide, and eventually emit at least a portion of the light collected by the solar collector toward at least some of the algae within the bioreactor 12.

In yet additional embodiments, the bioreactor system 10 comprises one or more light sources that can alternate between artificial and natural light. In such an embodiment, the system can be configured to utilize natural light during periods of solar light availability and automatically or manually switch to artificial light when insolation or solar output falls below a pre-determined level. Further, one, two or more light sources could perform both natural and artificial lighting or a first light source could provide the artificial light source, while a second light source could provide the natural light. Alternatively, the light source or sources may operate simultaneously at various levels to maximize light availability to an organism (e.g., algae).

In some embodiments, an agitation system is arranged in the bioreactor system 10 to agitate, circulate, or otherwise manipulate the water, algae, effluent nutrient stream, flue gases, or some combination thereof. The agitation system can be configured so that the algae is continually mixed, where at least some of the algae is exposed to light while other algae is not exposed to light (e.g., the other algae is placed into a dark cycle). The agitation system may operate to advantageously reduce an amount of light-providing surface area to a volume of the algae within the bioreactor 12, yet still obtain a desired amount of lipid production. Additionally or alternatively, light/dark cycling may be accomplished by turning the light source ON/OFF).

In various applications, a bioreactor system 10 comprising both a bioreactor 12 and an extraction system 224, and optionally a system for refining or processing biofuel 226, may be attached to a waste treatment facility such that the bioreactor system 10 utilizes an effluent stream from the waste treatment facility as a nutrient source for the algae, which is subsequently harvested for biofuel that may be utilized to power the waste treatment facility.

In other applications, a bioreactor system 10 comprising both a bioreactor 12 and an extraction system 224, and optionally a system for refining or processing biofuel 226, may be incorporated into an automobile, train, airplane, ship, or any other vehicle having an internal combustion engine. In such applications, the CO₂ produced by the engine may be utilized by, for example, a recovery system 216 as a nutrient source for the algae and the heat generated by the engine may be utilized to promote algal growth by, for example, incorporating thermoelectric devices to convert the heat into electricity to power the bioreactor light source and/or maintaining a desired temperature profile.

In other embodiments, a bioreactor system 10 comprising both a bioreactor 12 and an extraction system 224, and optionally a system for refining or processing biofuel 226, may be utilized in concert with a power plant. In such embodiments, the excess heat generated at the power plant may be utilized to heat and dry the harvested algae. In certain embodiments, particularly in embodiments wherein the harvested algae has a hydrocarbon content greater than about 70%, the harvested algae may be directly utilized as fuel in the power plant without the need for any extraction, refining, or processing.

In other embodiments, a bioreactor system 10 in the form of a portable bio-system comprising both a bioreactor 12 and an extraction system 224, and optionally a system for refining or processing biofuel 226, may be shipped to, dropped into, and/or delivered a disaster zone as a means of providing fuel for emergency use.

Although growing and harvesting algae (broadly referred to as biomass) for biofuel or biodiesel, feedstock, and/or other purposes has been generally known since at least the late 1960's, there has been a renewed interest in this technology in part because of rising petroleum costs. Microscopic algae (hereinafter referred to as micro-algae) are regarded as being superb photosynthesizers and many species are fast growing and rich in lipids, especially oils. Some species of micro-algae are so rich in oil that the oil accounts for over fifty percent of the micro-algae's mass. These and other interesting qualities and characteristics of micro-algae are discussed in, for example, “An Algae-Based Fuel” by Olivier Danielo, Biofutur, No. 255 (May 2005).

Two types of micro-algae that are generally known to produce a high percentage of oil are Botryococcus braunii (commonly abbreviated to “Bp”) and Diatoms. Diatoms are unicellular algae generally placed in the family Bacillariophyceae and are typically brownish to golden in color. The cell walls of Diatoms are made of silica.

There are approximately 100,000 known species of algae around the world and it is estimated that more than 400 new species are discovered each year. Algae are differentiated mainly by their cellular structure, composition of pigment, nature of the food reserve, and the presence, quantity, and structure of flagella. Algae phyla (divisions) include, for example, blue/green algae (Cyanophyta), euglenids (Euglenophyta), yellow/green and golden/brown algae (Chrysophyta), dinoflagellates and similar types (Pyrrophyta), red algae (Rhodophyta), green algae (Chlorophyta), and brown algae (Phaeophyta).

In the production of biofuel, it is known that micro-algae is faster growing and can synthesize up to thirty times more oil than other terrestrial plants used for the production of biofuel, such as rapeseed, soybean, oil palm, wheat, or corn. One of the main factors for determining the yield or productivity of biofuel from micro-algae is the amount of algae that is exposed to sunlight.

Many types of algae produce bi-products such as colorants, poly-unsaturated fatty acids, and bio-reactive compounds. These and other bi-products of algae may be useful in food products, pharmaceuticals, supplements, and herbs, as well as personal hygiene products. In one embodiment, the algal bi-product left over after lipid extraction is used to produce animal feed.

In some embodiments of the various embodiments of the systems, devices, and methods described herein, the algae utilized may be genetically modified to, for example, increase the oil content of the algae, increase the growth rate of the algae, change one or more growth requirements (such as light, temperature and nutritional requirements) of the algae, enhance the CO₂ absorption rate of the algae, enhance the ability of the algae to remove pollutants (e.g., nitrogen and phosphate compounds) from a waste effluent stream, increase the production of hydrogen by the algae, and/or facilitate the extraction of oil from the algae. See, e.g., U.S. Pat. Nos. 5,559,220; 5,661,017; 5,365,018; 5,585,544; 6,027,900; as well as U.S. Patent Application Publication No. 2005/241017.

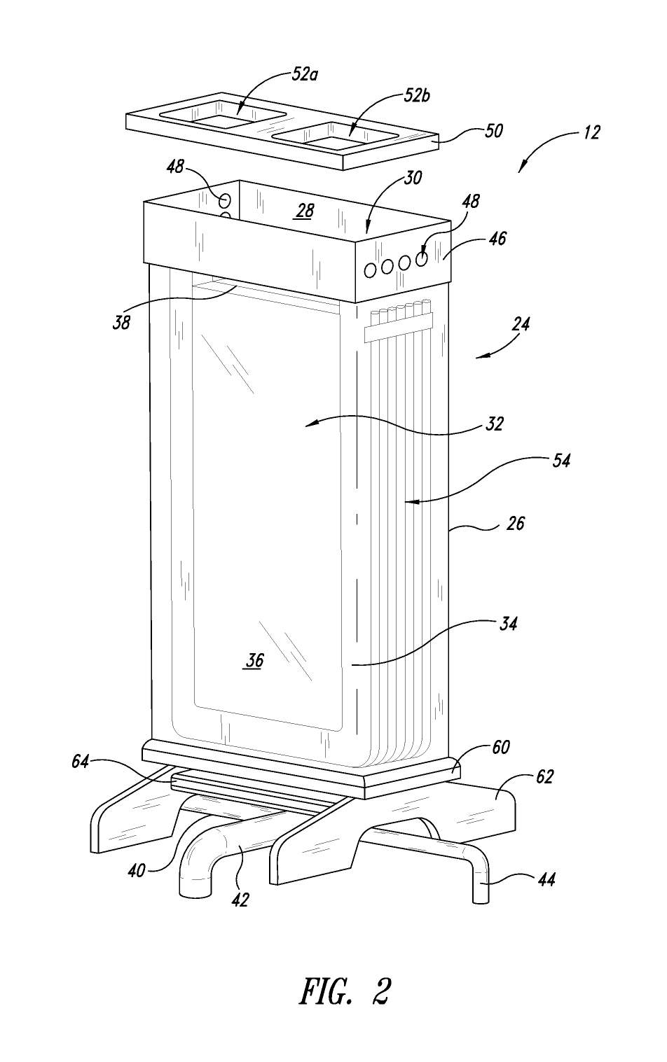

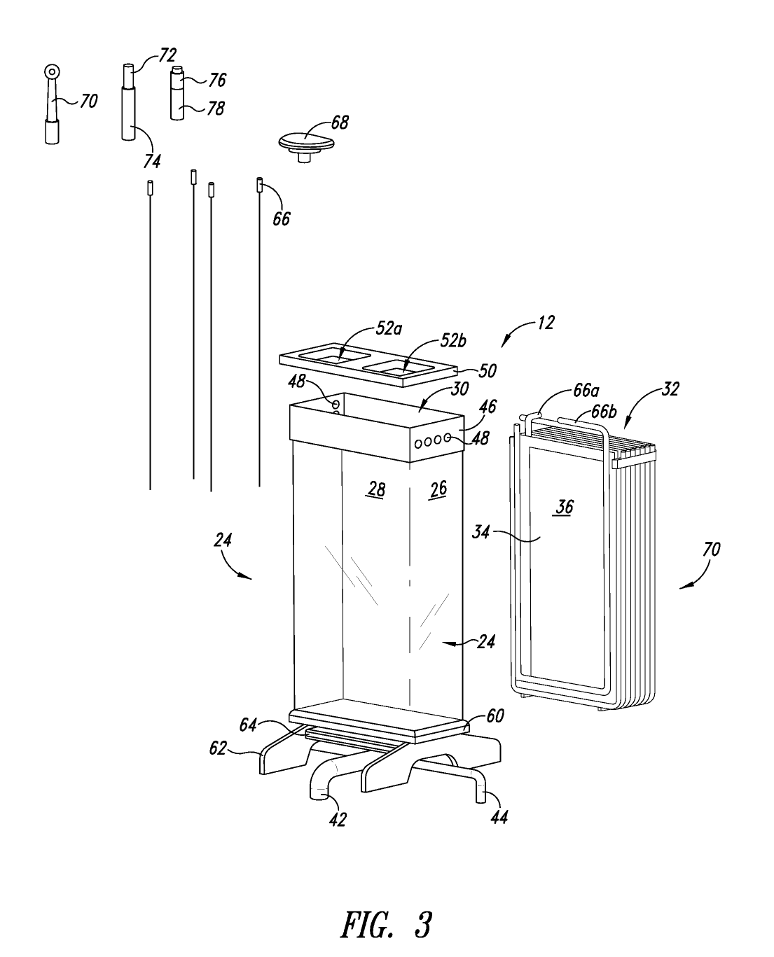

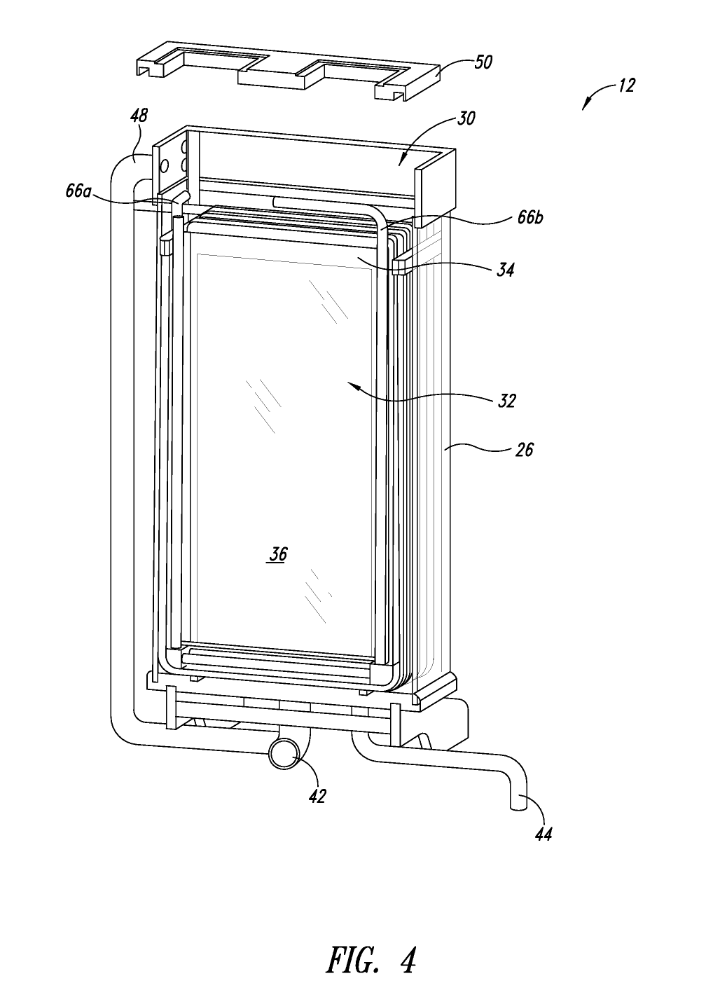

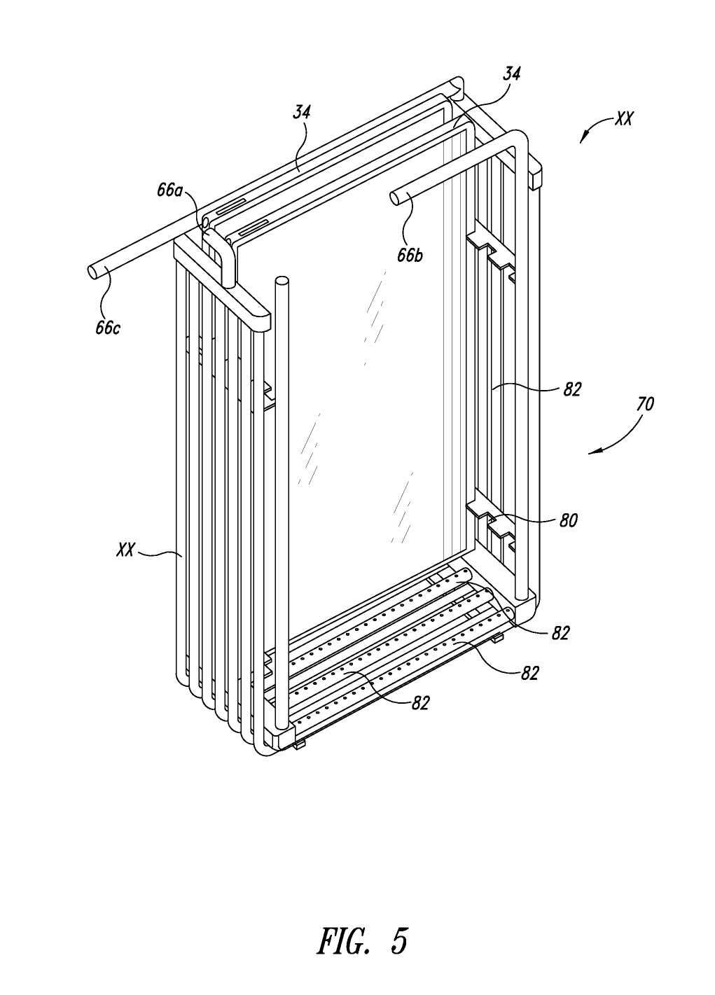

Referring to FIGS. 2, 3, 4, and 5, the bioreactor 12 may include at least one container 24 having and exterior surface 26 and an interior surface 28. In some embodiments, the interior surface 28 defines an isolated space 30 configured to retain biomasses, photosynthetic organisms, living cells, biological active substances, and the like. For example, the isolated space 30 defined by the interior surface 28 of the container 24 may be used to retain a plurality of photosynthetic organisms and cultivating media. The isolated space 30 can be a reservoir or collection region for holding biomass producing material.

The bioreactor 12 may take a variety of shapes, sizes, and structural configurations, as well as comprise a variety of materials. For example, the bioreactor 12 may take a cylindrical, tubular, rectangular, polyhedral, spherical, square, pyramidal shape, and the like, as well as other symmetrical and asymmetrical shapes. In some embodiments, the bioreactor 12 may comprise a cross-section of substantially any shape including circular, triangular, square, rectangular, polygonal, and the like, as well as other symmetrical and asymmetrical shapes. In some embodiments, the bioreactor 12 may take the form of an enclosed vessel 32 having one or more enclosures and/or compartments capable of sustaining and/or carrying out a chemical process such as, for example the cultivation of photosynthetic organisms, organic matter, a biochemically active substances, and the like.

Among the materials useful for making the container 24 of the bioreactor 12 examples include, translucent and transparent materials, optically conductive materials, glass, plastics, polymer materials, and the like, or combinations or composites thereof, as well as other materials such as stainless steel, Kevlar, and the like, or combinations or composites thereof. Further exemplary materials include concrete, (including, for example, concreter blocks, prestressed concrete, precasted concrete, pre-formed concrete, and the like), fiberglass, vinyl, polyvinyl chloride (PVC) plastic, metal, polyurethane foam, and the like, or other suitable building materials.

In some embodiments, the container 24 may comprise one or more transparent or translucent materials to allow light to pass from the exterior surface to a plurality of photosynthetic organisms and cultivation media retained in the isolated space 30. In some further embodiments, a substantial portion of the container 24 comprises a transparent or translucent material. Examples of transparent or translucent materials include glasses, PYREX® glasses, plexiglasses, acrylics, polymethacrylates, plastics, polymers, and the like or combinations or composites thereof.

The bioreactor 12 may also include a first lighting system 32. In some embodiments, the first lighting system 32 is received in the isolated space 30 of the container 24. The first lighting system 32 may comprise one or more light-emitting substrates 34. In some embodiments, each light-emitting substrate 34 has a first surface 36 and a second surface 38 opposite to the first surface. The one or more light-emitting substrates 34 may supply a first amount of light from the first surface 36 and a second amount of light from the second surface 38 to at least some of a plurality of photosynthetic organisms retained in the isolated space 30. In some embodiments, the one or more light-emitting substrates 34 are configured to provide at least a first and a second light-emitting pattern. The first lighting system 32 may further operate to produce at least a first illumination intensity level and a second illumination intensity level different than the first. In some embodiments, the second amount of light has at least one characteristic (e.g., light intensity, illumination intensity, light-emitting pattern, peak emission wavelength, on-pulse duration, and/or pulse frequency) different than a like characteristic of the first amount of light. In some other embodiments, the second amount of light has the same characteristics as the first amount of light.

In some embodiments, the bioreactor 12 may include one or more mirrored and/or reflective surfaces received in and/or formed on the interior 30 of the bioreactor 12. In some embodiments, a portion of the interior surface 28 of the bioreactor 12 may include mirrored and/or reflective surfaces such as, for example, a film, a coating, an optically active coating, a mirrored and/or reflective substrate, and the like. In some embodiments, the housing structures 14, 16 may include one or more mirrored and/or reflective surfaces in a portion adjacent to the exterior surface 26 of the container 24.

In some embodiments, the one or more mirrored and/or reflective surfaces may be configured to maximize distribution of light emitted by a lighting system 32.

The light-emitting substrates 34 may comprise a single light-emitting surface, or may comprise a multi-side arrangement with a plurality of light-emitting surfaces. The light-emitting substrates 34 may come in a variety of shapes and sizes. In some embodiments, the light-emitting substrates 34 may comprise a cross-section of substantially any shape including circular, triangular, square, rectangular, polygonal, and the like, as well as other symmetrical and asymmetrical shapes.

The one or more light-emitting substrates 34 may include a plurality of light emitting diodes (LEDs). LEDs including organic light-emitting diodes (OLEDs) come in a variety of forms and types including, for example, standard, high intensity, super bright, low current types, and the like. The “color” and/or peak emission wavelength spectrum of the emitted light generally depends on the composition and/or condition of the semi-conducting material used, and may include peak emission wavelengths in the infrared, visible, near-ultraviolet, and ultraviolet spectrum. Typically the LEDs' color is determined by the peak wavelength of the light emitted. For example, red LEDs have a peak emission ranging from about 625 nm to about 660 nm. Examples of LEDs colors include amber, blue, red, green, white, yellow, orange-red, ultraviolet, and the like. Further examples of LEDs include bi-color, tri-color, and the like. Emission wavelength may also depend on current delivered to the LEDs.

Certain biomasses, for example plants, algae, and the like comprise two types of chlorophyll, chlorophyll a and b. Each type typically possesses a characteristic absorption spectrum. In some cases the spectrum of photosynthesis of certain biomasses is associated with (but not identical to) the absorption spectra of, for example, chlorophyll. For example, the absorption spectra of chlorophyll a may include absorption maxima at about 430 nm and 662 nm, and the absorption spectra of Chlorophyll b may include absorption maxima at about 453 nm and 642 nm. In some embodiments, the one or more light-emitting substrates 34 may be configured to provide one or more peak emissions associated with the absorption spectra of chlorophyll a and chlorophyll b.

The plurality of LEDs may take the form of, for example, at least one LED array. In some embodiments, the plurality of LEDs may take the form of a plurality of two-dimensional LED arrays or at least one three-dimensional LED array.

The array of LEDs may be mounted using, for example, a flip-chip arrangement. A flip-chip is one type of integrated circuit (IC) chip mounting arrangement that does not require wire bonding between chips. Thus, wires or leads that typically connect a chip/substrate having connective elements can be eliminated to reduce the profile of the one or more light-emitting substrates 34.

In some embodiments, instead of wire bonding, solder beads or other elements can be positioned or deposited on chip pads such that when the chip is mounted upside-down in/on the light-emitting substrates 34, electrical connections are established between conductive traces of the light-emitting substrates 34 and the chip.

In some embodiments, the plurality of LEDs comprise a peak emission wavelength ranging from about 440 nm to about 660 nm, an on-pulse duration ranging from about 10 μs to about 10 s, and a pulse frequency ranging from about 1 μs to about 10 s.

In some embodiments, the one or more light-emitting substrates 34 include a plurality of optical waveguides to provide optical communication (e.g., optical coupling) between a source of light located in the exterior of the bioreactor 12 and a portion of the first lighting system 32 received in the isolated space 30.

The term “waveguide” generally refers to structures that guide waves, such as electromagnetic waves, light, sound waves, and the like. The term “optical waveguide” generally refers to any structure having the ability to guide optical energy. Examples of optical waveguides include lenses, mirrors, optical fibers, thin-film deposits, prisms, holograms, and the like. In some embodiments, the optical waveguides take the form of a plurality of optical fibers.

In some embodiments, the first lighting system 32 may further include at least one optical waveguide on the exterior surface 26 of the container 24 optically coupled to the first lighting system 32. The at least one optical waveguide may be configured to optically couple a source of solar energy to a portion of the first lighting system 32 received in the isolated space 30. The source of solar energy may include a solar collector and a solar concentrator optically coupled to the solar collector and the portion of the first lighting 32. The solar concentrator can be configured to concentrated solar energy provided by the solar collector and to provide the concentrated solar energy to the portion of the first lighting system 32 received in the isolated space 30.

In some embodiments, the one or more light-emitting substrates 34 are encapsulated in a medium having a first index (n₁) of refraction and the growth medium has a second index of refraction (n₂) such that the differences between n₁ and n₂, at a given wavelength selected from a spectrum ranging from about 440 nm to about 660 nm, is less than about 1. Examples of the medium having a first index (n₁) of refraction include mineral oil. Mineral oil may also serve to cool the LEDs and prevent water migration into the electronics, for instance in the event of a panel case seal failure.

In some embodiments, the control system 200 is configured to control at least one of a light intensity, illumination intensity, light-emitting pattern, peak emission wavelength, on-pulse duration, and/or pulse frequency associated with the light-emitting substrates 34 based on a measured optical density.

The one or more light-emitting substrates 34 may be configured to supply an effective amount of light to a substantial portion of the plurality of photosynthetic organisms retained in the isolated space 30. In some embodiments, an effective amount of light comprises an amount sufficient to sustain a biomass concentration having an optical density (OD) value greater than from about 0.1 g/l to about 15 g/l. Optical density may be determined by having an LED on the surface of one panel and an optical sensor directly opposite on the surface of another panel. Alternatively, the initial sensor may be a separate device inside the medium. For each algae species, samples of the growth are taken and a concentration level is determined by filtering the algae and weighing the results. Samples are taken at a minimum of three different concentration levels and those values are corresponded to the optical readings from between the panels or device inside the medium and an algorithm is created using the data. Optical density may then be monitored optically and manipulated with the control system 200.

In some embodiments, an effective amount of light comprises an amount sufficient to sustain a photosynthetic organism density greater than 1 gram of photosynthetic organism per liter of cultivation media. In some embodiments, an effective amount of light comprises an amount sufficient to sustain a photosynthetic organism density greater than 5 grams of photosynthetic organism per liter of cultivation media. In some further embodiments, an effective amount of light comprises an amount sufficient to sustain a photosynthetic organism density ranging from about 1 gram of photosynthetic organisms per liter of cultivation media to about 15 grams of photosynthetic organisms per liter of cultivation media. In yet some other embodiments, an effective amount of light comprises an amount sufficient to sustain a photosynthetic organism density ranging from about 10 grams of photosynthetic organisms per liter of cultivation media to about 12 grams of photosynthetic organisms per liter of cultivation media. In some embodiments, the bioreactor 12 may further include conductivity probe 70. The bioreactor system 10 may further include one or more sensors including dissolved oxygen sensors 72, 74, pH sensors 76, 78, a level sensor 68, CO₂ sensors, oxygen sensors, and the like. The bioreactor system 10 may also include one or more thermocouples 6. The bioreactor 12 may include, for example, inlet and/or outlet ports 48, and inlet and/or outlet conduits 40, 42, 44, for providing or discharging process elements, nutrients, gasses, biomaterials, and the like, to and from the bioreactor 12.

Growth media may be for freshwater, estuarine, brackish, or marine bacterial or algal species and/or other microorganisms or plankton. The growth media may consist of salts, such as sodium chloride and/or magnesium sulfate, macro-nutrients such as nitrogen and phosphorus containing compounds, micro-nutrients such as trace metals, for example iron and molybdenum containing compounds and/or vitamins, such as Vitamin B12. The growth media may be modified or altered to accommodate various species and/or to optimize various characteristics of the cultured species, such as growth rate, protein production, lipid production and carbohydrate production.

The bioreactor system 10 may further include a second lighting system adjacent to the exterior surface 26 of the container. The second lighting system may comprise at least one light-emitting substrate 34 configured to provide light to at least some of the plurality of photosynthetic organisms retained in the isolated space 30 and located proximate to a portion of the interior surface 26 of the container 24. In some embodiments, the second lighting system includes at least one light-emitting substrate located on one side of housing structure 14, and at least one light-emitting substrate located on one side of housing structure 16.

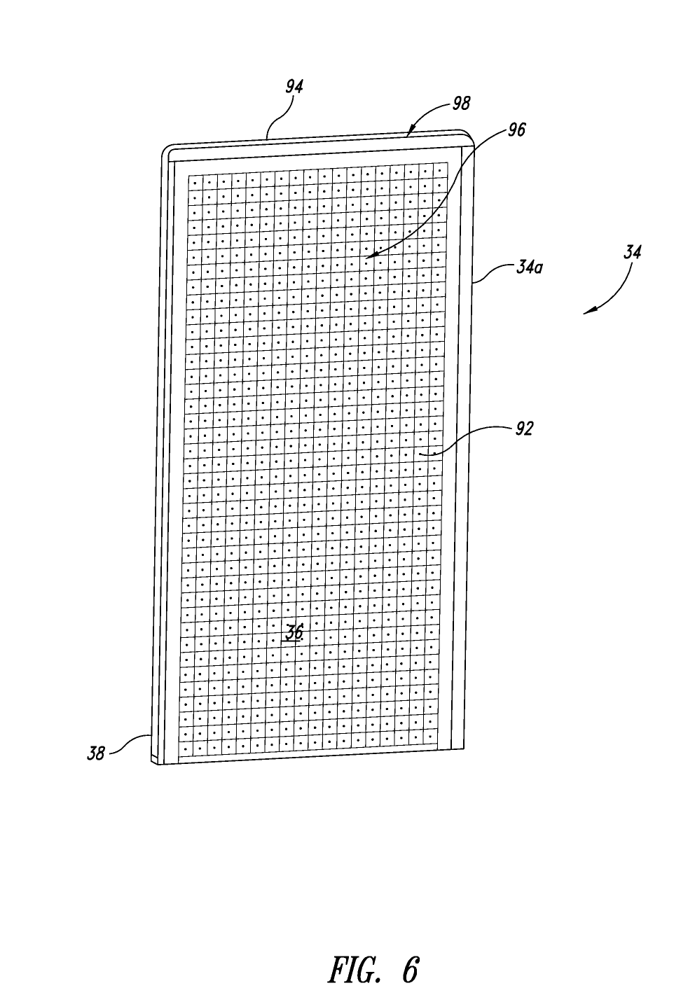

As shown in FIG. 6, in some embodiments, the one or more light-emitting substrates 34 take the form of light-energy-supplying substrates 34a having a first side 92 and a second side 94 opposite to the first side 92, the first and the second sides 92, 94 including one or more light-energy-supplying elements 92 that form part of a light-energy-supplying area 96. In some embodiments, each of the light-energy-supplying substrates 34a may be encapsulated, covered, laminated, and/or included in a medium having a first index (n₁) of refraction and the cultivation media has a second index of refraction (n₂) such that the differences between n₁ and n₂, at a given wavelength selected from a spectrum ranging from about 440 nm to about 660 nm, is less than about 1.

In some embodiments, the light-energy-supplying substrates 34a include a plurality of light sources 92 mounted to a flexible transparent base that forms part of the light-energy-supplying area 96. The light sources 92 can be wire bonded or mounted in a flip chip arrangement onto the flexible transparent base. In some embodiments, the light-energy-supplying substrates 34a may include a plurality of optical waveguides to provide optical coupling between a source light located in the exterior of the bioreactor 12 and the plurality of light-energy-supplying substrates received within the isolated space 30 of the bioreactor 12. In some embodiments, the light-emitting substrates 34 may be porous and hydrophilic.

In some embodiments, the bioreactor system 10 may take the form of a photosynthetic biomass cultivation system. The biomass cultivation system includes a control system 200 configured to automatically control at least one process variable associated with cultivating a photosynthetic biomass, and a bioreactor 12. The bioreactor 12 includes a structure 24 and a lighting system 32.

The structure 24 includes an exterior surface 26 and an interior surface 28, the interior surface 28 defines an isolated space 30 comprising a volume configured to retain the photosynthetic biomass suspended in cultivation media. The lighting system 32 is received in the isolated space 30 of the structure 24. In some embodiments, the lighting system 32 includes one or more light-emitting elements 34 including a light-emitting area 96 on each side of it sides 94, 98. The light-emitting area 96 forms part of a light-emitting-area 96 to bioreactor-volume interface. In some embodiments, the light-emitting area to bioreactor volume ratio ranges from about 0.005 m²/Liter to about 0.1 m²/Liter. The light-emitting elements may take the form of a plurality of two-dimensional LED arrays or at least one three-dimensional LED array.

The photosynthetic biomass cultivation system may include one or more sensors 212 operable to determine at least one of a temperature, pressure, light intensity, density, gas content, pH, fluid level, sparging gas flow rate, salinity, fluorescence, absorption, mixing, turbulence, and/or the like.

The control system 200 is configured to automatically control the at least one process variable selected from a bioreactor interior temperature, bioreactor pressure, pH level, nutrient flow, cultivation media flow, gas flow, carbon dioxide gas flow, oxygen gas flow, light supply, and/or the like.

In some embodiments, the bioreactor 12 comprises one or more effluent streams providing fluidic communication of gasses, liquids, and the like between the exterior and/or interior of the bioreactor 12. In some embodiments, the bioreactor 12 make take the form of enclosed system wherein no effluent streams go in or out on a continual basis.

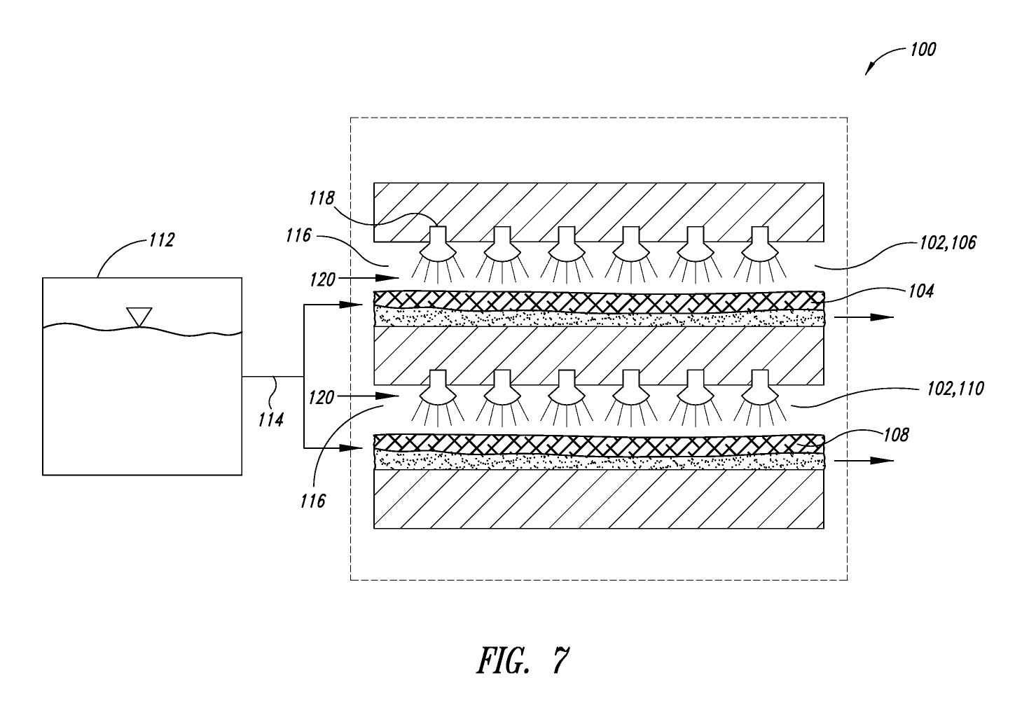

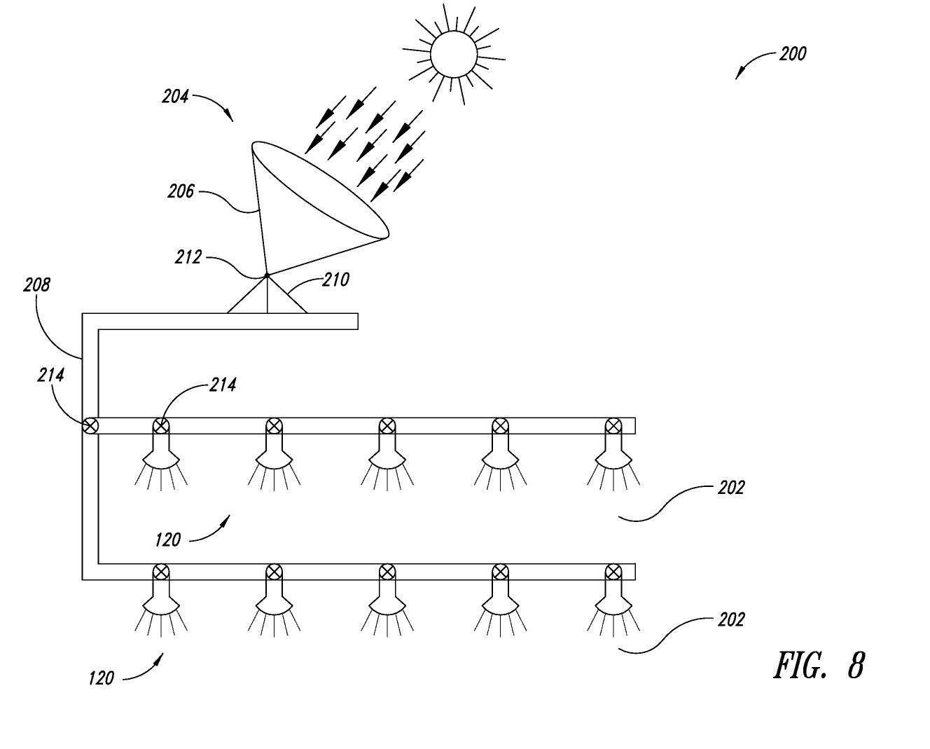

As shown in FIGS. 7 and 8, a bioreactor 100 may be configured to increase a light exposure of photosynthetic organisms located in the bioreactor 100. For example, the bioreactor 100 may include at least first level 106 of the bioreactor 100 for supporting a first surface layer 104 of photosynthetic organisms, and a second level 110 of the bioreactor 100 for supporting a second surface layer 108 of photosynthetic organisms. In some embodiments, the first level 106 is physically separated from the second level 110. In some embodiments, a structural partition positioned within the bioreactor 100 separates the respective levels 106, 110.

The bioreactor 100 may further include a lighting system comprising a number of light emitters 118 arranged to direct a first amount of light toward the first surface layer 104 of photosynthetic organisms and further arranged to direct a second amount of light toward the second surface layer 108 of photosynthetic organisms. In some embodiments, the first surface layer 104 of photosynthetic organisms comprises algae from a first phyla and the second surface layer 108 of photosynthetic organisms comprises algae from a second phyla. In some further embodiments, the first and second surface layers 104, 108 of photosynthetic organisms comprise algae from the same phyla.

The lighting system may include a plurality of LEDs. In some embodiments, the lighting system includes a plurality of fiber optic waveguides. The lighting system directs artificial and/or natural light toward the respective surface layers of photosynthetic organisms 104, 108 in the bioreactor 100.

In some embodiments, the lighting system is configured to direct natural light toward the respective surface layers 104, 108 of the photosynthetic organisms in the bioreactor 100. The bioreactor 100 may further include a solar collector system 204 to receive sunlight, wherein the lighting system directs at least a portion of the sunlight toward the respective surface layers 104, 108 of the photosynthetic organisms in the bioreactor 100.

For example, bioreactors 12, 100 can be an enclosed vessel in which a chemical process, for example photosynthesis, is carried out that involves organisms, organic matter, biochemically active substances, etc. In one embodiment, each bioreactor 12, 100 is a cylindrical device made of stainless steel, Kevlar, or an equivalent material. In another embodiment, each bioreactor 12, 100 is the triangular-shaped bioreactor, similar to the one produced by GreenFuels Technology Corporation of Cambridge, Mass., USA. In yet another embodiment, each bioreactor 12, 100 refers to a device or system for growing cells or tissues in the context of cell culture, such as the disposable chamber or bag, called a CELLBAG®, made by Panacea Solutions, Inc., and usable with systems developed by Wave Biotechs, LLC. In a further embodiment, each bioreactor 12, 100 can be a specially designed landfill for rapidly growing, transforming and/or degrading organic structures. In yet a further embodiment, each bioreactor 12, 100 comprises a sphere and a mirror located outside of the sphere, wherein the shape of the sphere maximizes the surface to volume ratio of the algae contained therein and the mirror reflects light, such as sunlight, into the sphere.

Bioreactor systems 10 are often required to operate under strict environmental conditions. Thus, there are many components, assemblies, and/or sub-systems that comprise the bioreactor system 10, for example sub-systems for controlling gasses (e.g., air, oxygen, CO₂, etc.) in and out of the bioreactor, effluent streams, flow rates, temperatures, pH balances, etc. It is understood that bioreactor systems 10 may employ a variety of sensors, controllers, mechanical agitators, and/or filtration systems, etc. These devices may be controlled and operated by a central control system. It is understood that the design and configuration of a bioreactor system 10 can be complex and varied depending on the location and/or purpose of the bioreactor 12.

In one embodiment, the bioreactor system 10 includes sub-systems and/or devices that cooperate to monitor and possibly control operational aspects such as the temperature, salinity, pH, CO₂ levels, O₂ levels, nutrient levels, and/or the light. In further aspects, the bioreactor system 10 may include the ability to increase or decrease each aspect or parameter individually or in any combination, for example, temperature may be raised or lowered, gas levels may be raised or lowered (e.g., CO₂, O₂, etc.), pH, nutrient levels, light, etc., may be raised or lowered. The light can be natural or artificial. Some general lighting control aspects include controlling the duration that the light operates on portions of the algae in the bioreactor 12, 100, cycling the light (to include periods of light and dark), for example artificial light, to extend the growth of the algae past daylight hours, controlling the wavelength of the light, and/or controlling the intensity of the light. These aspects, among others, are described in further detail below.

In some embodiments, the bioreactor 100 is operable for processing micro-algae. The bioreactor 100 may include a number at levels, channels, or tubes 1025 according to one illustrated embodiment. In various embodiments, levels 102 may comprise stackable algae panels. A first surface layer of micro-algae 104 is photosynthesized on a first level 106, a second surface layer of micro-algae 108 is photosynthesized on a second level 110, and so on. Although only two levels 102 are illustrated, it is understood that the bioreactor 100 may have “1-n” levels 102, where n is greater than 2.

In one embodiment, a source 112 directs a stream 114 of micro-algae to the bioreactor 100 where the micro-algae are directed to the different levels or channels 102. The micro-algae may be separated based on a number of criteria, such as the specific density, size, and/or type of micro-algae. In addition, flue gasses 116 rich in CO₂ may be directed into the bioreactor 100 to enrich the micro-algae and provide the necessary amount Of CO₂ for the photosynthetic process to occur, as well as to assist in removing CO₂ and other gases from the flue gas.

In another embodiment, the algae is seeded or pre-placed in the bioreactor 100. An effluent stream is directed into the bioreactor 100 to provide nutrients to the algae. The effluent stream can be a stream of wastewater as described above. Additionally or alternatively, flue gasses 116 rich in CO₂ may be directed into the bioreactor 100 to enrich the micro-algae and provide the necessary amount of CO₂ for the photosynthetic process to occur.

The levels or channels 102 of the bioreactor 100, in which the algae is cultivated, can have a variety of configurations and/or cross-sectional shapes. For example, a first level or channel may be narrow in places and wide in other places to control an amount of light penetration on the algae. For example, narrow levels or channels can be arranged to provide a dark cycle for the algae, whereas the wide levels or channels permit the algae to cover a larger surface area so that more of the algae is exposed to the light.

The photosynthetic process typically employs both dark and light cycles. Dark cycles allow the algae to process a photon of light. During the light cycle, the algae absorb photons of light. By way of example, once a photon of light is absorbed, which happens in a range of about 10⁻¹⁴ to 10⁻¹⁰ seconds, it takes approximately 10⁻⁶ seconds for the algae to perform photosynthesis and reset itself to be ready to absorb another photon. Accordingly, the levels or channels 102 and/or lighting system can be arranged in the bioreactor 100 to advantageously control the light and dark cycles to increase the photosynthetic efficiency of the algae therein.

In some embodiments, a number of light emitters 118 are arranged in the bioreactor 100 at various locations proximate the surface layers of micro-algae 104, 108. The light emitters 118 can be LEDs for projecting artificial light, such as visible, infrared, and/or ultraviolet light, toward the surface layers of micro-algae 104, 108. In one embodiment, the light emitters 118 are LEDs developed by Light Sciences Oncology. The LEDs are spaced, oriented, and/or otherwise configured to maximize the photosynthetic process in the micro-algae. For example, adjacently located LEDs may be arranged to direct light of various wavelengths at different angles, may be arranged circumferentially around the level or channel or 102, may have different diffusion and/or dispersion characteristics, different light intensities, and the like. Further, at least some light emitters 118 may be located within an interior portion or outside of an exterior portion of the level or channel 102. In some embodiments a number of light emitters 118 are arranged in the bioreactor 100 at various locations within the surface layers of micro-algae 104, 108.

In another embodiment, the light emitters 118 are fiber optic waveguides that receive artificial light from LED's, for example. In this embodiment, different banks of LEDs may provide light different wavelengths of light. Therefore a first set of fiber optic waveguides may receive light of a first wavelength while a second set of fiber optic waveguides may receive light of a second wavelength. The wavelength of the light emitted from the LEDs can be selected to at least approximately correspond to an absorption capacity of the algae to increase the photosynthetic and/or growth processes. Power for LEDs can come from a grid or from photovoltaic cells, as described below. Additional details regarding fiber optic waveguides and fiber optic networks generally, are provided in the discussions below regarding additional and/or alternate embodiments.

In yet another embodiment, the light emitters 118 are LEDs arranged on a sheet and the sheet is rolled to form the tube or channel 102 through which the algae are cultivated. Additionally or alternatively, the LEDs are arranged in transparent tubes or coils. These so-called light tubes are disposed longitudinally within the level or channel 102, so that as the algae flows through the level or channel (e.g., tube) 102 more algal cells will be exposed to the light from the number of light tubes. Consequently, this arrangement operates to increase the photosynthetic surface area of the algae in the bioreactor 100.

In another embodiment, a plurality of LEDs are coupled to or located outside of the level or channel 102 and oriented to direct light into the level or channel 102, which may, for example, be formed as one or more tubes. Additionally or alternatively, the level or channel 102 can be lined with a reflective coating on an interior surface thereof or made from a reflective material. Further, the heat generated by the LEDs could be routed through the bioreactor 100, as necessary, to algae and/or effluent stream.

FIG. 8 shows a bioreactor 200 for processing micro-algae within a number of levels or channels 202, according to one illustrated embodiment.

For purposes of brevity and clarity, are surface layers of micro-algae, the flue gasses, and the bioreactor structural features are not shown. The bioreactor 200 supports a solar collector system 204 for collecting sunlight and directing the light into the bioreactor 200. In one embodiment, the solar collector system 204 is coupled with a fiber optic cable system that is capable of receiving and routing sunlight into the bioreactor 200 as described in for example, U.S. Pat. No. 5,581,447.

In one embodiment, the solar collector system 204 includes an internal transparent cover to absorb light and to reflect infrared light or alternatively, a filter to substantially filter out undesired wavelengths of light, such as light having wavelengths in the infrared range of wavelengths. The cover or filter can be located within the solar collector housing 206, which may be located on top of, or proximate to, the bioreactor 200, according to one embodiment. In another embodiment, the solar collector housing 206 is located remotely from the bioreactor 200 and coupled to fiber optic cables or waveguides 208 that can be routed (e.g., underground) to the bioreactor 200. In addition, the solar collector system 204 includes a fixed portion 210 and a rotatable portion 212. The fixed portion 210 can be mounted to the bioreactor 200. The solar collector housing 206 is coupled to the rotatable portion 212 and is controllable to be rotated, tilted, and/or swiveled (e.g., up to six degrees of freedom) so that a desired amount of solar energy can be collected. The solar collector system 204 may be combined with any of the bioreactors disclosed herein.

A plurality of solar collector cells or photovoltaic cells are arranged in a frame within the housing 206 and oriented with respect to the transparent cover to receive the light passing through the transparent cover. Each solar collector cell includes a lens, such as a Fresnel lens, mounted to a mirrored, funnel shaped collector, which in turn is coupled to at least one fiber optic waveguide 208. The fiber optic waveguides 208 may be bundled or independently routed to different portions of the bioreactor 200 to selectively direct the light to the micro-algae located therein. In one embodiment, a light dispersion unit with a prismatic cover is coupled to an output end of the fiber optic waveguide 208 for selectively dispersing light toward a portion of the micro-algae.

Fiber optic waveguides 208 typically include a core surrounded by a cladding material, where the light propagates through the core. The core is typically made from transparent silica (e.g., glass) or a polymeric material (e.g., plastic). In one embodiment, the fiber optic waveguide 208 is made from a molecularly engineered electro-optic polymer that is commercially available from Lumera Corporation.

A control system 214 can be used to direct the light through the fiber optic waveguides 208 by selectively controlling a number of optical switches 214 arranged in the fiber optic network. The fiber optic switches 214 generally operate to re-direct, guide, and/or to block light traveling through the fiber optic network.

Optical switches can be generally classified into the following categories: (1) opto-mechanical switches, which include a micro-electrical mechanical system (MEMS) switches; (2) thermo-optical switches; (3) liquid-crystal and liquid-crystals-in-polymer switches; (4) gel/oil-based “bubble” switches; (5) electro-holographic switches; and others switches such as acousto-optic switches; semiconductor optical amplifiers (SOA); and ferromagnetic switches. The structure and operation of these optical switches are described in, for example, Amy Dugan et al., The Optical Switching Spectrum: A Primer on Wavelength Switching Technologies, Telecomm. Mag.; and Roland Lenz, Introduction to All Optical Switching Technologies, v.1, (Jan. 30, 2003).

It is understood and appreciated that the optical switches to be used with the solar collector system 204 may operate according to any of the aforementioned principals or may operate according to different principals. In one exemplary embodiment, the optical switch is an “Electroabsorption (EA) Optical Switch” developed by OKI® Optical Components Company. In another exemplary embodiment, the optical switch is an “Efficient Linearized Semiconductor Optical Switch” (ELSOM) developed by TRW, Inc. In yet another exemplary embodiment, the optical switch is a “Lithium Niobate (LiNbO₃) Optical Switch” developed by the Microelectronics Group of Lucent Technologies, Inc. In still yet another exemplary embodiment, the optical switch is a discrete, electro-optical switch developed by Lumera Corporation. The optical switches can include amplifiers or regenerators to condition the light, electrical signal, and/or optical signal.

The control subsystem 214 provides control signals to cause at least some of the fiber optic waveguides 208 to emit light at successively discrete times (e.g., scan the light over an area of algae) and/or emit light at varying intensities. It is understood that at least in one embodiment and at any discrete moment in time, at least one fiber optic waveguide 208 can be in a light-emitting state while another fiber optic waveguide 208 is in a non-light-emitting state. It should be appreciated that the control system 214 can be programmed to achieve a desired emission sequence of the light onto at least various portions of the micro-algae within the bioreactor 200.

In embodiments wherein the multiple layers of algae comprise stackable algae panels with CO₂ sparging as a nutrient feed and means for mixing, artificial lighting, such as LEDs contained within the panels or fiber optic feeds connected to a solar collector device, may be matched to the algal absorption spectrum. The panels may be stacked horizontally or vertically.

FIG. 9 shows an exemplary method 600 for providing light energy to a substantial portion of a plurality of photosynthetic organisms in liquid growth media within a bioreactor 12.

At 602, the method 600 includes providing a bioreactor containment structure 24 having an exterior surface 26 and an interior surface 28, the interior surface 28 defining an isolated space 30 configured to house a plurality of photosynthetic organisms and liquid growth media.

At 604, the method 600 includes providing a plurality of light-energy-supplying substrates 34. In some embodiments, the plurality of light-energy-supplying substrates 34 comprise a first side 36 and a second side 38 opposite to the first side 36. In some embodiments, the first and the second sides 36, 38 include one or more light-energy-supplying elements 92 that form part of a light-energy-supplying area 96, the plurality of light-energy-supplying substrates 34 is received within the isolated space 30 of the bioreactor 12.

In some embodiments, providing a plurality of light-energy-supplying substrates 34 comprises providing a sufficient amount of the one or more light-energy-supplying elements 92 that form part of a light-energy-supplying area 96, such that a ratio of light-energy-supplying area 96 to a volume of the isolated space of the bioreactor is greater than about 0.005 m²/Liter.

At 606, the method 600 further includes vertically mixing the photosynthetic organisms included in the liquid growth media. Vertical mixing may include using circulated air or mechanical agitators or stirring systems. The method 600 may further include axially mixing the photosynthetic organisms included in the liquid growth media. In some embodiments, the method 600 may further include agitating the photosynthetic organisms in liquid growth media during photosynthesis. In some embodiments, one or more gas spargers 82 are used to provide vertical and/or axial mixing of the photosynthetic organisms included in the liquid growth media.

At 608, the method 600 further includes supplying an effective amount of light energy from the light-energy-supplying substrates 34 to a substantial portion of the plurality of photosynthetic organisms in the bioreactor 12. In some embodiments, supplying an effective amount of light energy from the light-energy-supplying substrates 34 includes an amount sufficient to sustain a biomass concentration from about 0.1 g/l to about 17.5 g/l. In some embodiments, supplying an effective amount of light energy from the light-energy-supplying substrates 34 includes an amount sufficient to sustain a photosynthetic organism density greater than about 10 gram of photosynthetic organism per liter of cultivation media. The method 600 may further include stressing the photosynthetic organism to affect, for example, a lipid content. Examples of stressing include changing stress variable to induce nutrient deprivation, nitrogen-deficiency, silicon-deficiency, pH, CO₂ levels, oxygen levels, degree of sparging, or other conditions that affect growth and/or development of an organism, and the like. See e.g., Spoehr & Milner: 1949, Plant Physiology 24, 120-149. In particular, nitrogen deficiency reduced growth rates and resulted in high oil content: 1 Tornabene et al: 1983, Enzyme and Microbial Technology, 435-440; 2—Lewin: 1985, Production of hydrocarbons by micro-algae: isolation and characterization of new and potentially useful algal stains, SER1/CP-231-2700, 43-51; 3—Zhekisheva et al: 2002, Journal of Phycology, 325-331. Silicon deficiency in diatoms yielded similar results: Tadros & Johansen: 1988, Journal of Phycology, 445-452. In some embodiments, the method further includes temperature stressing the photosynthetic organism.

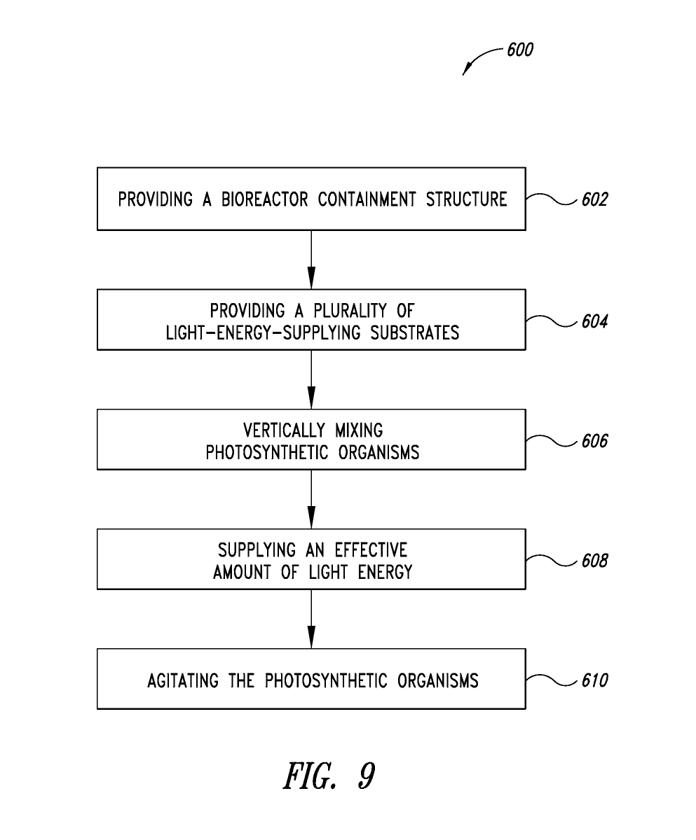

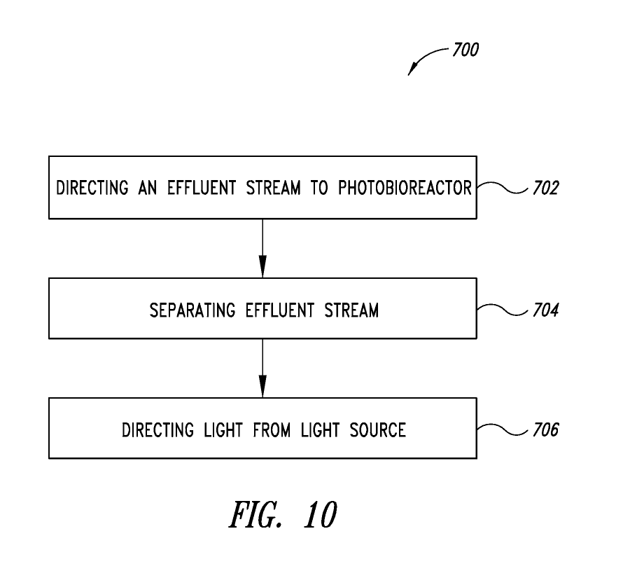

FIG. 10 shows an exemplary method 700 for increasing a ratio of light-emitting-area to a photobioreactor-volume interface of a photobioreactor.

At 702 the method 700 includes directing an effluent stream to the bioreactor 12. The photobioreactor 100 comprising a structure having an inner surface defining a photobioreactor volume.

At 704 the method 700 includes separating the effluent stream to direct one portion of the effluent stream to one region 106 of the bioreactor 100 having a first amount of algae 104 and to direct another portion of the effluent stream to another region 110 of the bioreactor 100 having a second amount of algae 108. In some embodiments, the effluent stream includes the first amount and the second amount of algae. In some embodiments, the first amount of algae 104 is a first type of algae and the second amount of algae 108 is a different type of algae.

At 706 the method 700 further includes directing light from a light source having a ratio of light-emitting-area to a photobioreactor-volume interface 120 of a bioreactor 100 toward at least some of the algae 104, 108 in the bioreactor 100 to encourage a photosynthetic reaction in the algae. Directing light from the light source may include directing natural light from a fiber optic network. Directing light from the light source may include directing light from a light-emitting diode (LED). The method 700 may further include receiving sunlight in a solar collector. In some embodiments, the method 700 may further include agitating the algae during photosynthesis.

In some embodiments, increasing a ratio of light-emitting-area to a photobioreactor-volume interface may further include increasing a light intensity per photosynthetic organism.

FIGS. 11-17 show open bioreactor systems 800, 840, 880 that can be similar to the closed bioreactor systems disclosed herein. Generally, open bioreactor systems 800, 840, 880 can be exposed to the surrounding environment. Natural resources (e.g., ambient gases such as air, ambient fluids such as rain water or runoff, sunlight, thermal energy, airflow, and the like) in the surrounding environment may be used to affect the production of biomass. The holding capacity, configuration (e.g., average depth of holding chamber or reservoir), and other processing parameters of the bioreactor systems 800, 840, 880 can be selected based on the desired biomass production rate and type of biomass producing material utilized. Accordingly, natural resources may be utilized to reduce manufacturing costs, improve the quality of the biomass, yield high production rates, and the like. Because a biomass producing material in open bioreactors can utilize natural resources, manufacturing costs of biomass passively produced in the open bioreactor may be less than manufacturing costs of biomass produced in closed bioreactors employing primarily actively delivered resources.

FIG. 11 shows an open-air bioreactor 800 filled with biomass producing material 806. The illustrated bioreactor 800 includes a reservoir 810 holding the biomass producing material 806 such that a sufficient amount of sunlight and a sufficient amount of ambient gases are exposed to the biomass producing material 806 to support a wide range of bioreactions (e.g., small to large scale bioreactions).

To reduce the manufacturing cost of the open bioreactor 800, the reservoir 810 can be a natural reservoir, such as a lake, pond, stream, or other naturally occurring body of water. Various types of additives can be disposed into the water to produce a desired biomass producing material. In some embodiments, the water can be drained from the reservoir 810 and replaced with biomass producing material.

With continued reference to FIG. 11, the shore 820 surrounding the upper surface 830 of the biomass producing material 806 defines an “opening” or exposure window 822. The biomass producing material 806 (e.g., algae) can utilize sunlight passing through the opening 822, although light systems or other auxiliary systems 824 can be added to the open bioreactor 800, if need or desired. For example, in some embodiments, the open bioreactor 800 may include a lighting system received in the reservoir 810. For example, in some embodiments, light systems or other auxiliary systems 824 may be received in the reservoir 810, and substantially held in place by, and/or suspended from, for example, a floating support 826, such as floating booms, floating dry docks, and the like. In some embodiments, the reservoir 810 is an artificial reservoir that can be formed at a location suitable for biomass production. Advantageously, the artificial reservoir 810 can be rapidly installed a wide variety of locations for convenient biomass production near, for example, a consumption site. For example, for a facility (e.g., a manufacturing plant) that consumes a significant amount of biomass product, the open bioreactor 800 can be installed near the facility to minimize or limit biomass transportation costs.

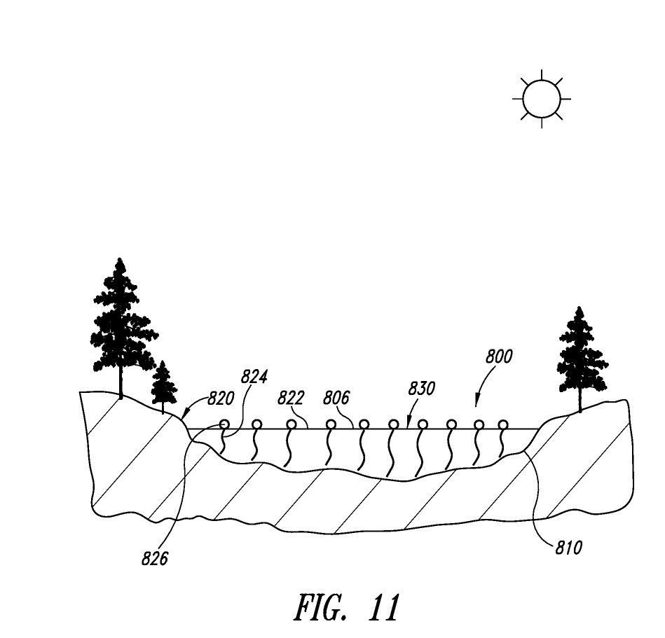

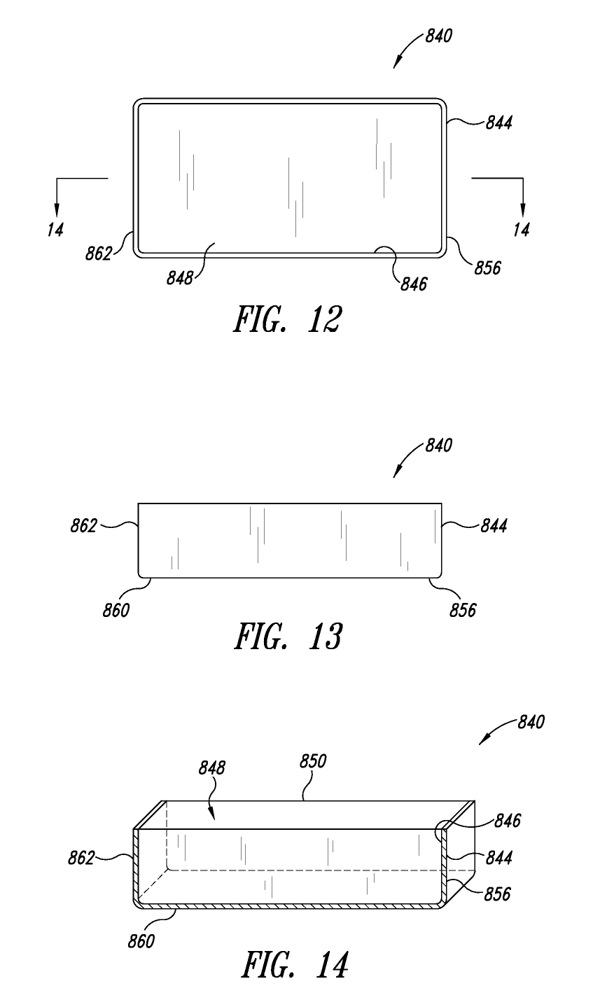

Referring to FIGS. 12-14, the bioreactor 840 is a portable open-air tank having an exterior surface 844 and an interior surface 846. The interior surface 846 defines a reservoir or chamber 848 for holding the biomass producing material. The bioreactor 840 includes an opening 850 through which biomass producing material or its components can be delivered. A wall 856 of the bioreactor 840 can comprise transparent or translucent materials to allow additional ambient light to reach the biomass producing material. As used herein, the term “wall” is broadly construed to include, without limitation, a bottom, sidewall, and other structures suitable for forming a reservoir or holding chamber. The illustrated wall 856 includes a bottom 860 and sidewall 862 extending away from the bottom 860.

The portable bioreactor 840 can be conveniently transported to a wide range of locations for on-site biomass production. The holding capacity of the chamber 848 can be selected based on biomass production rate. For example, the chamber 848 can hold a few gallons to thousands of gallons of biomass producing material. Additionally, the average depth, cross-sectional area (e.g., the cross-sectional area of the chamber 848 taken generally perpendicular to an upper surface of biomass producing material when the chamber 848 is filled), and other dimensions of the bioreactor 840 can be varied as desired.

An array of open and/or closed bioreactors can be used for a highly scalable biomass production system. The number and type of bioreactors can be periodically changed in order to efficiently make a desired amount of biomass product.

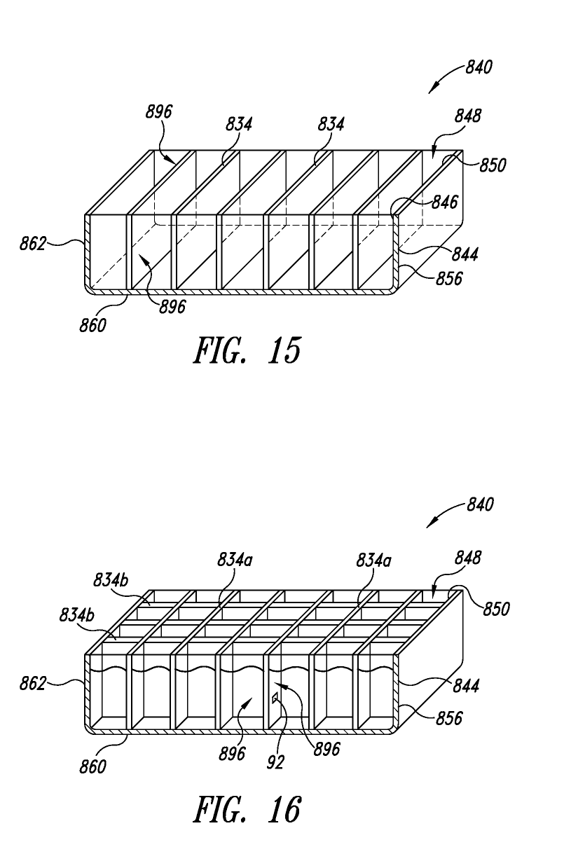

Various types of lighting systems can be employed with the bioreactors, such as the bioreactors 800, 840, 880. Referring to FIGS. 15 and 16, for example, the open bioreactor 840 may include a lighting system comprising one or more light-emitting substrates 834 each having a first surface and a second surface opposite to the first surface. The illustrated lighting system of FIG. 15 includes an array of spaced apart light-emitting substrates 834. The light-emitting substrates 834 are configured to supply a first amount of light from the first surface and a second amount of light from the second surface to at least some of a plurality of photosynthetic organisms retained in the reservoir 848. As shown in FIG. 16, the one or more light-emitting substrates 834 may take the form of light-energy-supplying substrates 834a, 834b including one or more light-energy-supplying elements 92 that form part of a light-energy-supplying area 896.

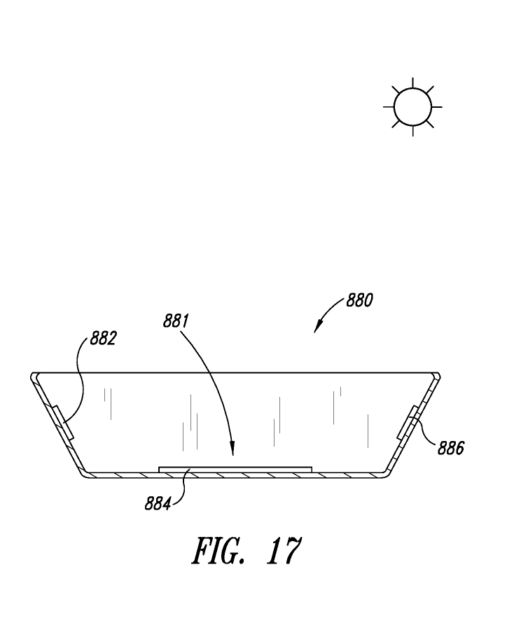

Additionally, various features, components, systems, and sub-systems described herein with respect to closed bioreactors can be incorporated into open bioreactors. For example, FIG. 17 shows an open bioreactor 880 with an auxiliary system 881 configured to actively affect biomass production. The illustrated auxiliary system 881 includes auxiliary production devices 882, 884, 886 spaced from one another. In contrast to passive bioreactors that utilize primarily natural resources, output from the auxiliary system 881 can be used to significantly adjust the production of the biomass. Each of the auxiliary production devices 882, 884, 886 can include one or more light sources (e.g., light-emitting substrates, waveguides, solar collectors, sensors, and other types of lighting systems), fluid delivery systems for delivering liquids and/or gases, drainage systems, control system, agitators (e.g., horizontal agitators suitable mixing biomass producing material disposed in horizontally oriented containers), and the like. For example, the auxiliary system 881, in some embodiments, includes one or more light sources that can controllably direct light to the biomass producing material.

In some embodiments, the one or more auxiliary production devices 882, 884, 886 may take the form of one or more light sources and may take any geometric form including but not limited to, for example, cylindrical, conical, regular, or irregular forms. In some embodiments, the one or more auxiliary production devices 882, 884, 886 may take a cylindrical geometric form having a cross-section of substantially any shape including but not limited to circular, triangular, square, rectangular, polygonal, and the like, as well as other symmetrical and asymmetrical shapes, or combinations thereof. In some embodiments, the one or more auxiliary production devices 882, 884, 886 may take the form of substantially conical structures or frusto-conical structures, as well as faceted structures including but not limited to prismatoids, polyhedrons, pyramids, prisms, wedges, and the like, or combinations thereof. In some embodiments, the one or more auxiliary production devices 882, 884, 886 may take the form of any of the disclosed light-emitting substrates. The light-emitting substrates may be carried, suspended, or provided by permanent, semi-permanent, and/or removably affixed structures. In some embodiments, the light-emitting substrates may be received within open bioreactor 880 and substantially held in place by, and/or suspended from, for example, a floating support 826, such as floating booms, floating dry docks, and the like.

In some embodiments, the one or more auxiliary production devices 882, 884, 886 may comprise an optical material adapted to transmit a desired amount of optical energy. Optical materials may include, without limitation transparent, translucent, or light-transmitting materials, or combinations or composites thereof. Suitable transparent, translucent, or light-transmitting materials include those materials that offer a low optical attenuation rate to the transmission or propagation of light waves. Examples of transparent, translucent, or light-transmitting materials include but are not limited to crystals, epoxies, glasses, borosilicate glasses, optically clear materials, semi-clear materials, plastics, thermo plastics, polymers, resins, thermal resins, and the like, or combinations or composites thereof.

In some embodiments, the one or more auxiliary production devices 882, 884, 886 may be coupled via one or more optical fibers to a source of solar energy including a solar collector. In some embodiments, the one or more auxiliary production devices 882, 884, 886 may be optically coupled to a solar collector system including a solar collector and a solar concentrator. The solar concentrator can be configured to concentrate solar energy provided by the solar collector and to provide the concentrated solar energy to at least a portion of the one or more auxiliary production devices 882, 884, 886.

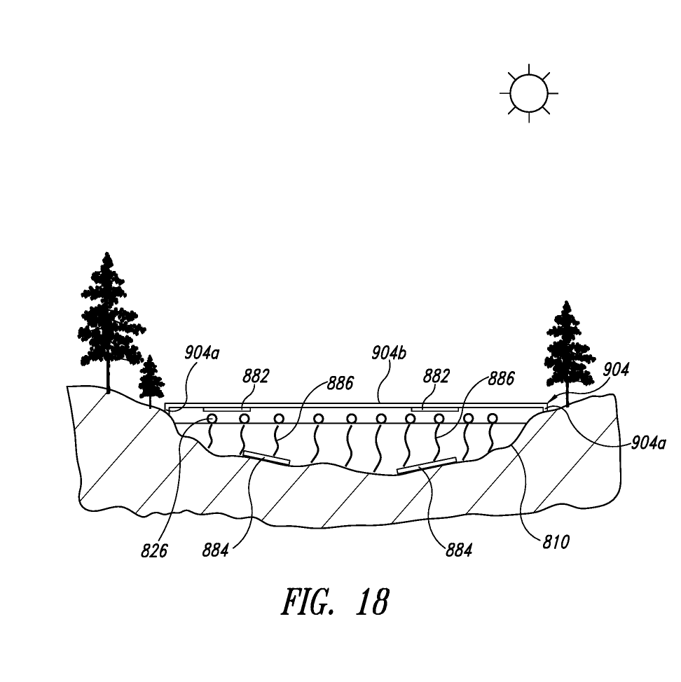

Referring to FIG. 18, in some embodiments, reservoirs 810 such as natural reservoirs (e.g., a lake, pond, stream, or other naturally occurring body of water) may be adapted to create a closed-system, a substantially closed-system, a partially closed-system, or variations thereof adapted for biomass production. In some embodiments, reservoirs 810 may be adapted to create a controlled environment system adapted for biomass production.

As previously noted, biomasses such as, for example, algal biomasses are often cultured in open-air systems (e.g., ponds, raceway ponds, lakes, natural reservoirs, artificial reservoirs, and the like, as well as regular and irregular shaped structures capable of sustaining biomass growth) that are subject to contamination, or are limited by the inability to substantially control the various process parameters (e.g., temperature, incident light intensity, flow, pressure, nutrients, and the like) involved in cultivating algae. Accordingly, some embodiments include systems, devices, and methods for environmental control of biomass production in open-air systems.

In some embodiments, for example, the reservoir 810 may include an isolator 904 configured to partially isolate, substantially isolate, completely isolate, or variations thereof the reservoir 810 from a surrounding open air environment. The illustrated isolator 904 includes supports 904a and cover 904b extending between the supports 904a. The cover 904b extends above and across the biomass in the reservoir 810.

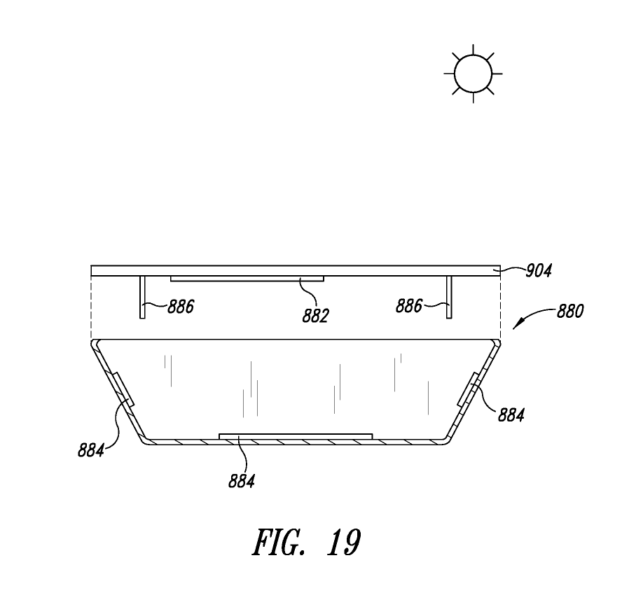

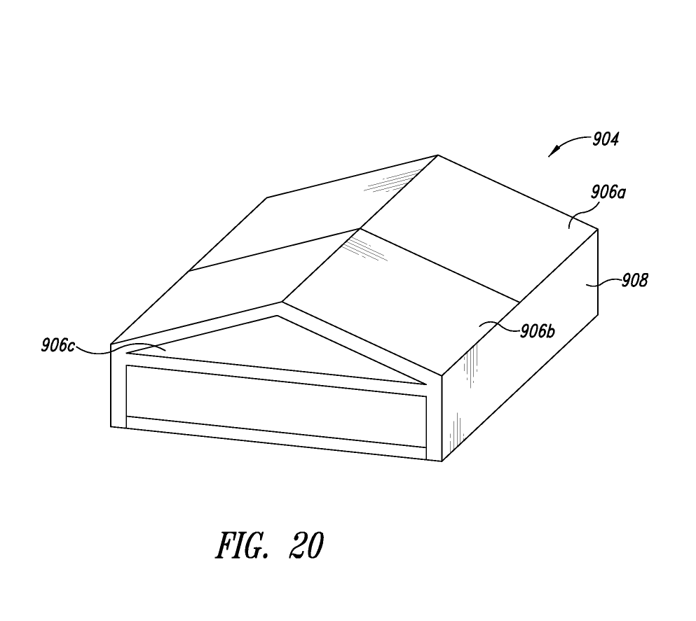

Referring to FIG. 19, in some embodiments, an open bioreactor 880 may be adapted to create a closed-system, a substantially closed-system, a partially closed-system, or variations thereof adapted for biomass production. For example, the bioreactor 880 may be adapted to include an isolator 904 configured to partially isolate, substantially isolate, completely isolate, or variations thereof the bioreactor 880 from a surrounding open air environment. In some embodiments, bioreactor 880 may be adapted to create a controlled environment system adapted for biomass production. In some embodiments, bioreactor 880 may include auxiliary production devices 882, 884, 886 spaced from one another. In contrast to passive bioreactors that utilize primarily natural resources, output from the auxiliary production devices 882, 884, 886 can be used to significantly adjust the production of the biomass.

Referring to FIG. 20, the isolator 904 may take any regular or irregular shape, and may have a cross-section of any suitable geometric form. The isolator 904 may be constructed of any suitable materials. The illustrated isolator 904 includes one or more panels 906a and cover 906b extending between supports 908. The isolator 904 may also include other support structures 906c configured to extend above and/or across a biomass in the bioreactor.

In some embodiments, the isolator 904 is configured to control one or more process parameters (e.g., temperature, incident light intensity, flow, pressure, nutrients, and the like) involved in cultivating algae. For example, the isolator 904 may include one or more structures, coatings, filters, operatives, masks, shades, panels, levers, or combinations thereof for controlling the amount of light (natural or artificial) passing through the isolator 904 and onto a biomass retained in a bioreactor. In some embodiments, the panels 906a, 906b may comprise an optical material (or other term from above) suitable to permit the passage of artificial or natural into the bioreactor.

In some embodiments, portions 906a, 906b, 906c, 908 of the isolator 904 may be configured to control the duration that the light operates on portions of, for example, an algal mass in the bioreactor, cycling the light (to include periods of light and dark), for example artificial light, to extend the growth of the algae past daylight hours, controlling the wavelength of the light, controlling the lighting patterns, and/or controlling the intensity of the light. For example, the panels 906a, 906b may be moved to adjust the amount of light, if any, that reaches the biomass. The supports 906c, 908 may further include vertical panels that can be moved to adjust the amount of light, if any, that reaches the biomass.

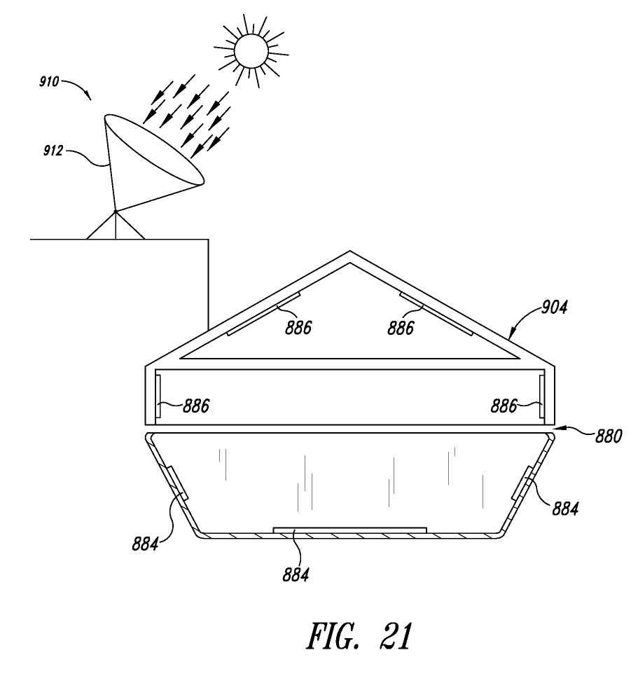

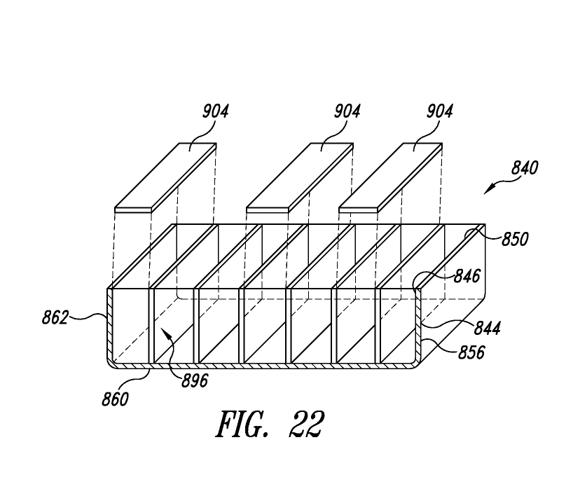

FIGS. 21, and 22 show various open bioreactors 840, 880 that have been modified to include one or more environment controlling structures 904. The one or more environment controlling structures 904 may be operable to partial isolate, substantially isolate, completely isolate, or variations thereof the various open bioreactors 840, 880 from an open air environment.

In some embodiments, the one or more environment controlling structures 904 may be configured to control one or more process parameters (e.g., temperature, incident light intensity, flow, pressure, nutrients, and the like) involved in cultivating algae. In some embodiments, the one or more environment controlling structures 904 may be configured to limit access of the biomass retained in the various open bioreactors 840, 880 from the outside.

Some open bioreactors 840, 880 may be limited in their ability to provide sufficient light to sustain dense populations of photosynthetic organisms cultivated within. Accordingly, in some embodiments, the structure 904 may include one or more auxiliary production devices 886 carried by the isolator 904. The auxiliary production devices 886 may be carried by various components of the structure 904, such as panels 906a, 906b and/or the support structures 906c, and 908. As previously noted, in some embodiments, the one or more auxiliary production devices 886 may take the form of any of the disclosed light-emitting substrates suitable to provide a sufficient amount of light to sustain dense populations of photosynthetic organisms cultivated within the bioreactors 840, 880.

In some embodiments, the environment controlling structures 904 may be optically coupled to a source of solar energy and/or optically coupled to a portion of the one or more auxiliary production devices 884 and/or 886 received within. The source of solar energy may include a solar collector 910 and a solar concentrator 912 optically coupled to the solar collector and the portion of the one or more auxiliary production devices 884 and/or 886. The solar concentrator can be configured to concentrated solar energy provided by the solar collector and to provide the concentrated solar energy to the one or more auxiliary production devices 884 and/or 886.

As illustrated in FIG. 22, the bioreactors 840, can be modified to include one or more environment controlling structures 904.

The various embodiments described above can be combined to provide further embodiments. All of the U.S. patents, U.S. patent application publications, U.S. patent applications, foreign patents, foreign patent applications, and non-patent publications referred to in this specification and/or listed in the Application Data Sheet are incorporated herein by reference, in their entirety, including but not limited to:

U.S. patent application Ser. No. 11/608,527, filed Dec. 8, 2006, U.S. Provisional Patent Application No. 60/749,243 filed Dec. 9, 2005, U.S. Provisional Patent Application No. 60/773,183 filed February 14, U.S. Pat. No. 5,581,447, and U.S. Pat. No. 5,637,207, are incorporated herein by reference, in their entirety.

Aspects of the various embodiments can be modified, if necessary, to employ systems, circuits, and concepts of the various patents, applications, and publications to provide yet further embodiments, including those patents and applications identified herein. While some embodiments may include all of the light systems, reservoirs, containers, and other structures discussed above, other embodiments may omit some of the light systems, reservoirs, containers, or other structures. Still other embodiments may employ additional ones of the light systems, reservoirs, containers, and structures generally described above. Even further embodiments may omit some of the light systems, reservoirs, containers, and structures described above while employing additional ones of the light systems, reservoirs, containers generally described above.

As one of skill in the art would readily appreciate, the present disclosure comprises systems, devices and methods incorporating light sources to cultivate and/or grow biomasses, photosynthetic organisms, living cells, biological active substances, and the like, by any of the systems, devices and/or methods described herein.

These and other changes can be made in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A bioreactor system for cultivating photosynthetic organisms, comprising:
a container having an exterior surface and an interior surface, the interior surface defining an isolated space configured to retain a plurality of photosynthetic organisms and cultivation media; and
a first lighting system comprising one or more light-emitting substrates received in the isolated space of the container, each having a first surface and a second surface opposite to the first surface, the one or more light-emitting substrates configured to supply a first amount of light from the first surface and a second amount of light from the second surface to at least some of a plurality of photosynthetic organisms retained in the isolated space.

2. The bioreactor system of claim 1 wherein the second amount of light has at least one characteristic that has a value that is different than a value of a characteristic of the first amount of light.

3. The bioreactor system of claim 2 wherein the at least one characteristic is at least one of a light intensity, an illumination intensity, a light-emitting pattern, a peak emission wavelength, an on-pulse duration, and/or a pulse frequency.

4. The bioreactor system of claim 1 wherein the second amount of light has at least one characteristic that is the same as at least one characteristic of the first amount of light.

5. The bioreactor system of claim 1 wherein the one or more light-emitting substrates are configured to supply an effective amount of light to a substantial portion of the plurality of photosynthetic organisms retained in the isolated space.

6. The bioreactor system of claim 5 wherein an effective amount of light comprises an amount sufficient to sustain a biomass concentration having an optical density (OD) value greater than from about 0.1 g/l to about 17.5 g/l.

7. The bioreactor system of claim 5 wherein an effective amount of light comprises an amount sufficient to sustain a photosynthetic organism density greater than 1 gram of photosynthetic organism per liter of cultivation media.

8. The bioreactor system of claim 5 wherein an effective amount of light comprises an amount sufficient to sustain a photosynthetic organism density greater than 5 grams of photosynthetic organism per liter of cultivation media.

9. The bioreactor system of claim 5, wherein an effective amount of light comprises an amount sufficient to sustain a photosynthetic organism density ranging from about 1 gram of photosynthetic organisms per liter of cultivation media to about 15 grams of photosynthetic organisms per liter of cultivation media.

10. The bioreactor system of claim 5 wherein an effective amount of light comprises an amount sufficient to sustain a photosynthetic organism density ranging from about 10 grams of photosynthetic organisms per liter of cultivation media to about 12 grams of photosynthetic organisms per liter of cultivation media.

11. The bioreactor system of claim 1 wherein the one or more light-emitting substrates are configured to provide an amount of light comprising one or more peak emissions associated with the absorption spectra of chlorophyll a and chlorophyll b

12. The bioreactor system of claim 1 wherein the one or more light-emitting substrates each include a respective plurality of light-emitting diodes (LEDs).

13. The bioreactor system of claim 12 wherein the plurality of LEDs comprise:
a peak emission wavelength ranging from about 440 nm to about 660 nm;
an on-pulse duration ranging from about 1 μs to about 10 s; and
a pulse frequency ranging from about 1 μs to about 10 s.

14. The bioreactor system of claim 1 wherein the one or more light-emitting substrates each include a respective plurality of light-emitting diodes (LEDs) in the form of at least one LED array.

15. The bioreactor system of claim 1 wherein the first lighting system includes a source of light and a plurality of optical waveguides for optically coupling the source of light located externally with respect to the container to the one or more light-emitting substrates received in the isolated space of the container.

16. The bioreactor system of claim 1 wherein the first lighting system includes a plurality of optical fibers.

17. The bioreactor system of claim 1 wherein the first lighting system is operable to provide:
at least a first illumination intensity level and a second illumination intensity level different that the first illumination intensity level; and is configured to provide at least a first and a second light-emitting pattern from the one or more light-emitting substrates.

18. The bioreactor system of claim 1 wherein the first lighting system further comprises:
at least one optical waveguide on the exterior surface of the container for optically coupling a source of solar energy to the one or more light-emitting substrates received in the isolated space.

19. The bioreactor system of claim 1 wherein the first lighting system further comprises:
a solar collector; and
a solar concentrator optically coupled to the solar collector to provide concentrated solar energy from the solar collector to the at least one light-emitting substrate received in the isolated space.

20. The bioreactor system of claim 1 wherein the one or more light-emitting substrates are encapsulated in a medium having a first index (n₁) of refraction and the cultivation media has a second index of refraction (n₂) such that the differences between n₁ and n₂, at a given wavelength selected from a spectrum ranging from about 440 nm to about 660 nm, is less than about 1.

21. The bioreactor system of claim 1, further comprising:
a controller configured to control at least one of a light intensity, an illumination intensity, a light-emitting pattern, a peak emission wavelength, an on-pulse duration, or a pulse frequency supplied by the light-emitting substrates based on a measured optical density of the photosynthetic organisms and cultivation media.

22. The bioreactor system of claim 1, further comprising:
one or more sensors operable to detect at least one of a temperature, a pressure, a light intensity, an optical density, a gas content, a pH, a fluid level, or a sparging gas flow rate; and
a controller configured to control at least one of an illumination intensity, an illumination pattern, a peak emission wavelength, an on-pulse duration, and a pulse frequency based on the sensed at least one of the temperature, the pressure, the light intensity, the optical density, the gas content, the pH, the fluid level, or the sparging gas flow rate.

23. The bioreactor system of claim 1 wherein the photosynthetic organisms are selected from a group comprising prokaryotic algae and eukaryotic algae.

24. The bioreactor system of claim 1 wherein the photosynthetic organisms are selected from one or more micro-algae.

25. The bioreactor system of claim 1, further comprising:
at least one gas source in fluid communication with the isolated space.

26. The bioreactor system of claim 1, further comprising:
a second lighting system adjacent to the exterior surface of the container, the second lighting system comprising at least one light-emitting substrate configured to provide light to at least some of the plurality of photosynthetic organisms retained in the isolated space and located proximate to a portion of the interior surface of the container.

27. The bioreactor system of claim 1 wherein a substantial portion of the container comprises a transparent or translucent material that allows light to pass from the exterior surface to the plurality of photosynthetic organisms and cultivation media retained in the isolated space.

28. The bioreactor system of claim 1 wherein a substantial portion of the container comprises transparent or translucent material selected from glasses, PYREX® glasses, plexi-glasses, acrylics, polymethacrylates, plastics, polymers, or combinations or composites thereof.

29. The bioreactor system of claim 1 wherein the container comprises an open air tank defining at least one opening to the isolated space such that a sufficient amount sunlight and a sufficient amount of ambient gases, external to the container, can pass through the at least one opening and to the plurality of photosynthetic organisms.

30. A method of operating a bioreactor system for providing light energy to a substantial portion of a plurality of photosynthetic organisms in liquid growth media within a bioreactor, comprising:
providing a plurality of photosynthetic organisms and a cultivation media to an isolated space defined by an interior surface of a bioreactor containment structure;
vertically mixing the photosynthetic organisms included in the liquid growth media; and
operating a plurality of light-energy-supplying substrates received within the isolated space of the bioreactor, each of the light-energy supplying substrates having a first side and a second side opposite to the first side, the first and the second sides including one or more light-energy-supplying elements that form part of a light-energy-supplying area to supply an effective amount of light energy from the light-energy-supplying substrates to a substantial portion of the plurality of photosynthetic organisms in the isolated space.

31. The method of claim 30 wherein operating a plurality of light-energy-supplying substrates includes operating a plurality of LEDs to deliver a peak emission wavelength ranging from about 440 nm to about 660 nm, an on-pulse duration ranging from about 1 μs to about 10 s, and a pulse frequency ranging from about 1 μs to about 10 s.

32. The method of claim 30 wherein operating a plurality of light-energy-supplying substrates includes operating at least one light source externally located to the isolated space to provide light to the plurality of light-energy-supplying substrates via a plurality of optical waveguides optically coupled between the at least one light source and the plurality of light-energy-supplying substrates received within the isolated space of the bioreactor.

33. The method of claim 30 wherein operating a plurality of light-energy-supplying substrates includes operating the plurality of light-energy-supplying substrates each comprising a surface coating having a first index (n₁) of refraction such that a difference between the first index (n₁) of refraction and a second index of refraction (n₂) from the cultivation media at a given wavelength selected from a spectrum ranging from about 440 nm to about 660 nm, is less than about 1.

34. The method of claim 30 wherein operating a plurality of light-energy-supplying substrates comprises operating a sufficient number of the one or more light-energy-supplying substrates that form part of a light-energy-supplying area, such that a ratio of light-energy-supplying area to a volume of the isolated space of the bioreactor containment structure is greater than about 0.005 m²/Liter.

35. The method of claim 30 wherein operating a plurality of light-energy-supplying substrates to supply an effective amount of light energy from the light-energy-supplying substrates includes operating the plurality of light-energy-supply substrates to supply an amount sufficient to sustain a biomass concentration having an optical density (OD) value greater than from about 0.1 g/l to about 17.5 g/l.

36. The method of claim 30 wherein operating a plurality of light-energy-supplying substrates to supply an effective amount of light energy from the light-energy-supplying substrates includes operating the plurality of light-energy-supplying substrates to supply an amount sufficient to sustain a photosynthetic organism density greater than about 10 gram (dry mass) of photosynthetic organism per liter of cultivation media.

37. The method of claim 30, further comprising:
axially mixing the photosynthetic organisms included in the liquid cultivation media.

38. The method of claim 30, further comprising:
agitating the photosynthetic organisms in liquid cultivation media during photosynthesis.

39. A photosynthetic biomass cultivation system, comprising:
a controller configured to automatically control at least one process variable associated with cultivating a photosynthetic biomass; and
a bioreactor comprising:
a structure having an exterior surface and an interior surface, the interior surface defining an isolated space configured to retain the photosynthetic biomass suspended in cultivation media; and
a lighting system comprising one or more light-emitting elements including a light-emitting area received in the isolated space of the structure, the light-emitting area forming part of a light-emitting-area to reactor-volume interface.

40. The system of claim 39 wherein a ratio of the light-emitting area to bioreactor volume ranges from about 0.005 m²/L to about 0.1 m²/L.

41. The system of claim 39 wherein the one or more light-emitting elements take the form of a plurality of two-dimensional arrays of light-emitting diodes (LEDs).

42. The system of claim 39 wherein the one or more light-emitting elements take the form of at least one three-dimensional array of light-emitting diodes (LEDs).

43. The system of claim 39, further comprising:
one or more sensors operable to sense at least one of a temperature, a pressure, a light intensity, a density, a gas content, a pH, a fluid level, a sparging gas flow rate, a salinity, a fluorescence, an absorption, a mixing, or a turbulence.

44. The system of claim 39 wherein the at least one process variable includes at least one of a bioreactor interior temperature, a bioreactor pressure, a pH level, a nutrient flow, a cultivation media flow, a gas flow, a carbon dioxide gas flow, an oxygen gas flow, or a light supply.

45. A bioreactor system configured to adjust a light exposure of photosynthetic organisms located in the bioreactor, the bioreactor comprising:
at least a first level to support a first surface layer of photosynthetic organisms;
a second level to support a second surface layer of photosynthetic organisms, the first level physically separated from the second level; and
a lighting system arranged to direct a first amount of light toward the first surface layer of photosynthetic organisms and further arranged to direct a second amount of light toward the second surface layer of photosynthetic organisms.

46. The bioreactor system of claim 45 wherein the lighting system is configured to provide the first amount of light having at least one characteristic having a first value suitable for growth of algae from a first phyla and the second surface having a second value that is configured to provide the second amount of light having at least one characteristic suitable for growth of algae from a second phyla, the second value different than the first value.

47. The bioreactor system of claim 45 wherein the first level is physically separated from the second level by a structural partition.

48. The bioreactor system of claim 45 wherein the lighting system includes a plurality of light-emitting diodes (LEDs).

49. The bioreactor system of claim 45 wherein the lighting system includes a plurality of fiber optic waveguides.

50. The bioreactor system of claim 45 wherein the lighting system directs artificial light toward the respective surface layers of photosynthetic organisms in the bioreactor.

51. The bioreactor system of claim 45 wherein the lighting system directs natural light toward the respective surface layers of the photosynthetic organisms in the bioreactor.

52. The bioreactor system of claim 45, further comprising:
a solar collector system coupled to the lighting system, the solar collector configured to receive sunlight; wherein the lighting system optically couples at least a portion of the received sunlight toward the respective surface layers of the photosynthetic organisms in the bioreactor vessel.

53. A method for increasing a ratio of light-emitting-area to a photobioreactor-volume interface of a photobioreactor, the method comprising:
directing an effluent stream to the photobioreactor, the photobioreactor comprising a structure having an inner surface defining a photobioreactor volume;
separating the effluent stream to direct one portion of the effluent stream to a first region of the photobioreactor, the first region holding a first amount of algae and to direct another portion of the effluent stream to a second region of the photobioreactor, the second region holding a second amount of algae; and
directing light from a light source toward at least some of the algae in the bioreactor to encourage a photosynthetic reaction in the algae, the light source comprising one or more light-emitting elements including a first and a second light-emitting area, the first and the second light-emitting areas forming part of a light-emitting-area to photobioreactor-volume interface.

54. The method of claim 53 wherein the effluent stream includes the first amount and the second amount of algae.

55. The method of claim 53 wherein the first amount of algae is a first type of algae and the second amount of algae is a different type of algae.

56. The method of claim 53, further comprising:
agitating the algae during photosynthesis.

57. The method of claim 53 wherein directing light from the light source includes directing natural light via a fiber optic network.

58. The method of claim 53 wherein directing light from the light source includes directing light from a light-emitting diode (LED).

59. The method of claim 53, further comprising:
receiving sunlight in a solar collector.

60. The method of claim 53 wherein directing the effluent stream further includes directing the effluent stream through auxiliary delivery system in the bioreactor, which is in the form of a reservoir.

61. A bioreactor system for producing biofuel from algae, the system comprising:
a bioreactor vessel;
a lighting system retained within the bioreactor vessel and arranged to direct an amount of light on at least some algae located within the bioreactor vessel, the lighting system respectively oriented within the bioreactor vessel to increase a photosynthetic process of the algae;
a control system configured to monitor and/or control at least one environmental condition within the bioreactor vessel; and
a light source optically coupled to the lighting system.

62. The bioreactor system of claim 61, further comprising:
an extraction system coupled to the bioreactor vessel to extract lipid, a medical compound, and/or a labeled compound from the algae within the bioreactor vessel.

63. The bioreactor system of claim 62 wherein the extraction system includes at least one press operable to urge the lipid from the algae.

64. The bioreactor system of claim 62 wherein the extraction system includes at least one centrifuge.

65. The bioreactor system of claim 62 wherein the extraction system comprises an extractant injection system operable to inject an extraction selected from the group consisting of: chemical solvents, supercritical gases or liquids, hexane, acetone, liquid petroleum products, and primary alcohols.

66. The bioreactor system of claim 61, further comprising:
a conversion system operable to convert the lipid to biofuel, wherein the conversion system receives the lipid from the extraction system.

67. The bioreactor system of claim 66 wherein the conversion system employs a transesterification catalyst and an alcohol.

68. The bioreactor system of claim 61, further comprising:
a temperature sensor positioned within a first region of the bioreactor vessel.

69. The bioreactor system of claim 68 wherein the control system monitors an output signal of the temperature sensor.

70. The bioreactor system of claim 61, further comprising:
an optical density measurement device to measure a concentration of the algae.

71. The bioreactor system of claim 61 wherein the light source comprises a plurality of light-emitting diodes.

72. The bioreactor system of claim 61 wherein the light source comprises a solar collector.

73. The bioreactor system of claim 61 wherein the lighting system comprises a network of fiber optic waveguides and optical switches, wherein the network is coupled to the solar collector.

74. The bioreactor system of claim 61, further comprising:
at least one or more filters arranged to filter particulates from an effluent stream containing at least some of the algae.

75. A method of cultivating algae in a bioreactor, the method comprising:
placing a first species and a second species of algae together in a portion of the bioreactor, wherein the first species includes a first light absorption capacity and the second species includes a second light absorption capacity; and
controllably directing light toward the first and the second species of algae.

76. The method of claim 75 wherein directing the light includes directing light having a first wavelength.

77. The method of claim 77 wherein directing light having a first wavelength includes controllably selecting the wavelength of the light to increase a number of photosynthetic reactions in the first and the second species of algae.
